SEQUENCE LISTING (45 sequences)

SEQ ID NO: 1 - DNA, 39 bp, Artificial Sequence
  ORF example
atggttataaatttggagtgtgaaggttattgcgtgtaa

SEQ ID NO: 2 - DNA, 33 bp, Artificial Sequence
  “Church-tag” tag sequence (forward direction)
gttataaatttggagtgtgaaggttattgcgtg

SEQ ID NO: 3 - DNA, 33 bp, Artificial Sequence
  “Church-tag” tag sequence (reverse direction)
cacgcaataaccttcacactccaaatttataac

SEQ ID NO: 4 - DNA, 36 bp, Artificial Sequence
  dyfhC1 primer
gatcggatccaaattccagttagccatgatgcggtc

SEQ ID NO: 5 - DNA, 54 bp, Artificial Sequence
  dyfhC2 primer
cacgcaataaccttcacactccaaatttataaccattatacacggacgctatgc

SEQ ID NO: 6 - DNA, 55 bp, Artificial Sequence
  dyfhC3 primer
gttataaatttggagtgtgaaggttattgcgtgacggattaattttgtttctctt

SEQ ID NO: 7 - DNA, 37 bp, Artificial Sequence
  dyfhC4 primer
gatcgtcgacgcgctcgatatcaccgatgaacaaccg

SEQ ID NO: 8 - DNA, 25 bp, Artificial Sequence
  cyfhC1 primer
caatccgctgctttatttctgtcag

SEQ ID NO: 9 - DNA, 25 bp, Artificial Sequence
  cyfhC2 primer
ttataacgaaatcaacgggaaacct

SEQ ID NO: 10 - DNA, 34 bp, Artificial Sequence
  dyggV1 primer
gatcggatccctctaaaaaataaggaattaaagg

SEQ ID NO: 11 - DNA, 56 bp, Artificial Sequence
  dyggV2 primer
cacgcaataaccttcacactccaaatttataaccataggatacctaattaattaac

SEQ ID NO: 12 - DNA, 54 bp, Artificial Sequence
  dyggV3 primer
gttataaatttggagtgtgaaggttattgcgtgaagagcgccatttcccaccgt

SEQ ID NO: 13 - DNA, 36 bp, Artificial Sequence
  dyggV4 primer
gatcgtcgactcatattgctgataacccgctgcggt

SEQ ID NO: 14 - DNA, 24 bp, Artificial Sequence
  cyggV1 primer
gttgacggccaggccaacagtcat

SEQ ID NO: 15 - DNA, 22 bp, Artificial Sequence
  cyggV2 primer
ataaccctgggcaatcgcctcg

SEQ ID NO: 16 - DNA, 484 bp, Escherichia coli
atgcgaattggacacggttttgacgtacatgcctttggcggtgaaggcccaattatcattggtggcgtacgcattccttacgaaaaaggattgctggcgcattctgatggcgacgtggcgctccatgcgttgaccgatgcattgcttggcgcggcggcgctgggggatatcggcaagctgttcccggataccgatccggcatttaaaggtgccgatagccgcgagctgctacgcgaagcctggcgtcgtattcaggcgaagggttatacccttggcaacgtcgatgtcactatcatcgctcaggcaccgaagatgttgccgcacattccacaaatgcgcgtgtttattgccgaagatctcggctgccatatggatgatgttaacgtgaaagccactactacggaaaaactgggatttaccggacgtggggaagggattgcctgtgaagcggtggcgctactcattaaggcaacaaaatgattga

SEQ ID NO: 17 - DNA, 537 bp, Escherichia coli
atgcgccgcgcttttataaccggagttttctttttgtctgaagtcgaatttagccacgaatactggatgcgtcacgcgctgacgctggcgaaacgtgcctgggatgagcgggaagtgccggtcggcgcggtattagtgcataacaatcgggtaatcggcgaaggctggaaccgcccgattggtcgccatgatcccaccgcacatgcagaaatcatggccctgcggcagggtggtctggtgatgcaaaattatcgtctgatcgacgccacgttgtatgtcacgcttgaaccatgtgtaatgtgtgccggagcgatgatccacagtcgcattggtcgcgtggtctttggtgcgcgtgacgcgaaaactggcgctgcgggatctttaatggatgtgctgcatcatccgggtatgaatcaccgagtggaaattacggaaggaatactggcggatgagtgcgcggcgttgctcagtgacttctttcgcatgcgccgccaggaaattaaagcgcagaaaaaagcgcaatcctcgacggattaa

SEQ ID NO: 18 - DNA, 621 bp, Escherichia coli
atgaggtatatagttgccttaacgggaggcattggcagtggcaagagtaccgttgccaatgcgtttgctgatctcggaattaacgtcattgatgccgatattattgcgcgtcaggtggttgaaccaggtgcacctgcgctacatgccattgctgatcactttggcgctaacatgattgctgctgatggaacattgcagcgccgggccttgcgcgagcggatcttcgccaacccggaagagaaaaactggcttaacgccctgctgcatccgctgattcagcaagagacgcaacaccagatccagcaagctacttccccctatgtactgtgggttgtgccattgctggtagaaaactcactgtataaaaaagcgaatcgagtgcttgtggtggatgtcagcccagaaacgcaacttaagcgcaccatgcagcgcgatgatgtaactcgcgagcatgtcgaacaaatccttgctgctcaggcaacgcgcgaagcccgccttgccgtggcagatgacgtcattgataataacggcgcaccggatgctatcgcatcggatgttgcccgcctgcacgcacactatttgcagcttgcgtcgcagtttgtctcacaggaaaaaccgtaa

SEQ ID NO: 19 - DNA, 852 bp, Escherichia coli
atgcggacacagtggccctctccggcaaaacttaatctgtttttatacattaccggtcagcgtgcggatggttaccacacgctgcaaacgctgtttcagtttcttgattacggcgacaccatcagcattgagcttcgtgacgatggggatattcgtctgttaacgcccgttgaaggcgtggaacatgaagataacctgatcgttcgcgcagcgcgattgttgatgaaaactgcggcagacagcgggcgtcttccgacgggaagcggtgcgaatatcagcattgacaagcgtttgccgatgggcggcggtctcggcggtggttcatccaatgccgcgacggtcctggtggcattaaatcatctctggcaatgcgggctaagcatggatgagctggcggaaatggggctgacgctgggcgcagatgttcctgtctttgttcgggggcatgccgcgtttgccgaaggcgttggtgaaatactaacgccggtggatccgccagagaagtggtatctggtggcgcaccctggtgtaagtattccgactccggtgatttttaaagatcctgaactcccgcgcaatacgccaaaaaggtcaatagaaacgttgctaaaatgtgaattcagcaatgattgcgaggttatcgcaagaaaacgttttcgcgaggttgatgcggtgctttcctggctgttagaatacgccccgtcgcgcctgactgggacaggggcctgtgtctttgctgaatttgatacagagtctgaagcccgccaggtgctagagcaagccccggaatggctcaatggctttgtggcgaaaggcgctaatctttccccattgcacagagccatgctttaa

SEQ ID NO: 20 - DNA, 696 bp, Escherichia coli
atgcgacttgataaatttatcgcacagcaactcggcgttagccgtgctattgccgggcgtgaaatccgcggcaatcgtgtcaccgtcgatggcgaaatcgtccgtaatgcagcgttcaaactgcttcctgaacatgatgtcgcttacgatggcaacccgctggcgcagcaacacggtccacgttacttcatgctcaataagcctcagggctatgtttgctccacggacgaccctgatcacccaacggtgctctattttcttgatgaaccggtagcgtggaaactgcatgcggcggggcggttggatattgataccaccggtctggtgctgatgactgatgatggtcagtggtcgcaccgcattacttctccgcgccatcattgcgagaagacctatctggtgacactggaatcacctgtagctgacgatacggcagagcaatttgctaaaggcgtgcagctgcataacgaaaaagatctcactaagcctgcggtgctggaagtgattaccccaacgcaggttcgtctgaccatcagcgaagggcgttatcatcaggtgaaacgcatgttcgccgccgtgggtaaccacgtggttgagctgcatcgtgaacgtattggcggtattacgctggatgctgatttagcccccggtgaatatcgtccgttaactgaagaagaaattgccagcgtcgtctaa

SEQ ID NO: 21 - DNA, 885 bp, Escherichia coli
atgattatgccgcaacatgaccaattacatcgctatctgtttgaaaactttgccgtgcgcggcgaactggtaaccgtttcggaaaccctgcaacagatccttgagaaccacgattatccgcagcccgttaaaaacgtgctggcagaactgctggttgcgaccagcctgttaaccgctacgctgaagtttgatggtgatatcaccgtacagctgcagggcgacggtccgatgaatctggcggttattaacggtaacaataaccagcagatgcgcggtgtggcgcgcgtgcagggcgaaattccagaaaatgccgacctgaaaacgctggtcggcaatggttacgtggtgatcaccattaccccgagcgaaggcgaacgctatcagggcgtagttggtctggaaggtgataccctggcggcctgcctggaagattactttatgcgttctgaacagctgccgacgcgcctgtttattcgcaccggcgacgtagacggcaaaccggctgcaggcggtatgttgttgcaggtaatgcctgcgcaaaatgcccagcaggacgactttgaccacctggcgacgctaaccgaaaccatcaaaaccgaagaactgctgaccttaccggcaaacgaagtgttgtggcgtttgtatcacgaagaagaggtgacggtttacgatccgcaggatgtggagttcaaatgcacctgctcgcgtgaacgttgcgccgatgcgctgaaaacgctgcctgatgaagaagttgatagcatcctggcggaagatggcgaaattgacatgcattgtgattactgcggtaaccactatctgttcaatgcgatggatattgctgaaatccgcaacaacgcgtctccggcagatccgcaagttcattaa

SEQ ID NO: 22 - DNA, 759 bp, Escherichia coli
gtggggagacgacgcggatttttaactatgcgtatcccccgcatttatcatcctgaaccactgaccagccattctcacatcgcgctttgcgaagatgccgccaaccatatcgggcgcgtactgcgcatggggccggggcaggcgttgcaattgtttgacggtagcaaccaggtctttgacgccgaaattaccagcgccagcaaaaaaagcgtggaagtgaaggtgctggaaggccagatcgacgatcgcgaatctccgctgcatattcacctcggtcaggtgatgtcgcgtggtgaaaaaatggaatttactatccagaaatcgatcgaactcggtgtaagcctcattacgccacttttttctgagcgctgcggcgttaaactggatagtgaacgtctgaacaagaagcttcagcagtggcagaagattgcaattgctgcctgtgagcagtgtggtcgtaaccgggtgccggaaatccgtccagcgatggatctggaagcctggtgtgcagagcaggatgaaggactgaaactgaatcttcacccgcgcgccagtaacagcatcaatacgttgccgttaccggttgaacgcgtccgcctgctgattggcccggaaggcggtttatcggcagatgaaattgccatgactgcccgctatcaatttactgatatcctgttgggacctcgcgttttgcgtacagagacaactgcgctcaccgccattaccgcgctacaagtacgatttggcgatttgggctaa

SEQ ID NO: 23 - DNA, 462 bp, Escherichia coli
atgatgaatcgagtaattccgctccctgatgagcaggcaacattagacctgggcgagcgggtagcgaaagcctgcgatggcgcaaccgtaatctatctgtatggcgatttaggcgcaggtaaaaccacctttagccggggctttttacaggctctgggtcatcagggtaatgtcaaaagccccacttatacgctggtcgaaccctatacgctcgacaacttaatggtctatcactttgatttgtaccgccttgccgatcccgaggagctggagtttatggggatccgcgattattttgccaacgatgccatctgcctggtggagtggccacaacaaggtacaggtgttcttcctgacccggatgtcgaaatacacattgattatcaggcacaaggccgtgaggcgcgcgtgagtgcggtttcctctgcgggtgaattgttgctggcgcgtttagccggttaa

SEQ ID NO: 24 - DNA, 987 bp, Escherichia coli
atgaaattacgctctgtaacctacgcattattcattgctggcctggctgcattcagcacatcttctctggcggcacaatctttacgtttcggttatgaaacatcacaaaccgactcgcaacatattgcggcgaaaaaattcaatgatttattgcaggagagaaccaaaggcgagctgaaattaaaactgttcccggacagcactctcggtaacgcgcaggcgatgatcagcggcgtacgtggcggcaccatcgatatggaaatgtccggctcgaataactttgccgggttatcaccagtgatgaacttgcttgatgtccctttcctgttccgcgataccgctcacgcgcataaaacgctcgacggcaaagtcggtgatgatctgaaagcctcacttgaaggtaaaggactgaaagtactggcctactgggaaaacggctggcgcgatgtcaccaactcgcgcgcaccggttaaaacccccgccgacctgaaagggctgaaaatccgcaccaacaatagcccgatgaatatcgccgcattcaaagtctttggcgctaacccgatcccgatgccgtttgccgaagtctataccgggctggaaacccgcactatcgacgctcaggaacacccgatcaacgtcgtctggtcagcaaaatttttcgaagtgcagaagttcctttctctgacgcaccacgcctattccccgcttctggtggtgatcaacaaagcgaagtttgatggcttaagtccggagttccagcaggcgctagtttcatctgcacaagaagcgggtaactatcagcgcaaactggttgctgaagatcagcaaaaaatcatcgacggcatgaaagaagcgggcgtggaagtcatcaccgatctcgaccgcaaagcctttagcgacgcactggggaatcaggttcgcgacatgtttgttaaagatgtgccgcagggagctgatctgctgaaagccgtggatgaggtgcaataa

SEQ ID NO: 25 - DNA, 573 bp, Escherichia coli
gtgaataataacctgcaaagagacgctatcgcagctgcgatagatgttctcaatgaagaacgtgtcatcgcctatccaacggaagccgttttcggtgttgggtgcgatcctgatagcgaaacagcagtgatgcgactgttggagttaaaacagcgtccggttgataaggggctgattttaatcgcagcaaattacgagcagcttaaaccctatattgatgacaccatgttgactgacgtgcagcgtgaaaccattttttcccgctggccaggtcctgtcacctttgtctttcccgcgcctgcgacaacaccgcgctggttgacgggccgctttgattcgcttgctgtacgagtcaccgaccatccgttggtggttgctttgtgccaggcttatggtaaaccgctggtttctaccagtgccaacttgagtggattgccaccttgtcgaacagtagacgaagttcgcgcacaatttggcgcggcgttcccggttgtgcctggtgaaacgggggggcgtttaaatccttcagaaatccgcgatgccctgacgggtgaactgtttcgacaggggtaa

SEQ ID NO: 26 - DNA, 459 bp, Escherichia coli
gtgggcttgtccacattagagcaaaaattaacagagatgattactgcgccagttgaggccctgggttttgaactggttggcatcgaatttattcgcggtcgcacatccacactgcgcatctatattgatagtgaagatggcatcaatgttgatgattgtgctgatgtgagccaccaggtaagtgctgtgctggatgttgaagatcccatcaccgttgcttataacctggaagtctcctcaccgggtctcgatcgcccactgttcacggctgaacactacgcccgttttgtcggagaagaggtgactctggttctccgtatggcggtacaaaaccgtcgtaaatggcagggcgttatcaaagcggtagacggtgaaatgatcacagttaccgtcgaaggtaaagatgaagtgttcgcgctgagtaatatccagaaggcgaacctggttccccacttttaa

SEQ ID NO: 27 - DNA, 711 bp, Escherichia coli
atggcaaccactcatttggatgtttgcgccgtggttccggcggccggatttggccgtcgaatgcaaacggaatgtcctaagcaatatctctcaatcggtaatcaaaccattcttgaacactcggtgcatgcgctgctggcgcatccccgggtgaaacgtgtcgtcattgccataagtcctggcgatagccgttttgcacaacttcctctggcgaatcatccgcaaatcaccgttgtagatggcggtgatgagcgtgccgattccgtgctggcaggtctgaaagccgctggcgacgcgcagtgggtattggtgcatgacgccgctcgtccttgtttgcatcaggatgacctcgcgcgattgttggcgttgagcgaaaccagccgcacgggggggatcctcgccgcaccagtgcgcgatactatgaaacgtgccgaaccgggcaaaaatgccattgctcataccgttgatcgcaacggcttatggcacgcgctgacgccgcaatttttccctcgtgagctgttacatgactgtctgacgcgcgctctaaatgaaggcgcgactattaccgacgaagcctcggcgctggaatattgcggattccatcctcagttggtcgaaggccgtgcggataacattaaagtcacgcgcccggaagatttggcactggccgagttttacctcacccgaaccatccatcaggagaatacataa

SEQ ID NO: 28 - DNA, 468 bp, Escherichia coli
atgagtcaggtgatcctcgatttacaactggcatgtgaagataattccgggttaccggaagagagccagtttcagacatggctgaatgcggtgatcccgcagtttcaggaagaatcggaagtgacgattcgcgtggtcgataccgccgaaagccacagtctgaatctgacctatcgcggtaaggataagccgaccaacgtgctctccttcccgtttgaagtgccgcctggcatggaaatgtcgctactgggcgatctggttatctgccgtcaggtggttgagaaggaagctcaggagcaaggcaaaccactggaggcgcactgggcgcatatggtggtgcacggcagtctgcatttgttaggttacgatcacatcgaagatgacgaagcagaagaaatggaagccctcgaaacagagattatgcttgctctgggctatgaggatccgtacattgccgagaaagaataa

SEQ ID NO: 29 - DNA, 1119 bp, Escherichia coli
atgcataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataa

SEQ ID NO: 30 - DNA, 480 bp, Escherichia coli
atgcaaaaacgggcgatttatccgggtactttcgatcccattaccaatggtcatatcgatatcgtgacgcgcgccacgcagatgttcgatcacgttattctggcgattgccgccagccccagtaaaaaaccgatgtttaccctggaagagcgtgtggcactggcacagcaggcaaccgcgcatctggggaacgtggaagtggtcgggtttagtgatttaatggcgaacttcgcccgtaatcaacacgctacggtgctgattcgtggcctgcgtgcggtggcagattttgaatatgaaatgcagctggcgcatatgaatcgccacttaatgccggaactggaaagtgtgtttctgatgccgtcgaaagagtggtcgtttatctcttcatcgttggtgaaagaggtggcgcgccatcagggcgatgtcacccatttcctgccggagaatgtccatcaggcgctgatggcgaagttagcgtag

SEQ ID NO: 31 - DNA, 699 bp, Escherichia coli
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactttgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgttgtcgtacgcgccatctccgacgtggccgatcaacagtctcatcttagcttcgatgagttcctggctgttgccgctaaacagtccagcctgatggttgagtcactggtgcagaaacttgcacatggctaa

SEQ ID NO: 32 - DNA, 1344 bp, Escherichia coli
gtgagcaatctgtcgctcgatttttcggataatacttttcaacctctggccgcgcgtatgcggccagaaaatttagcacagtatatcggccagcaacatttgctggctgcggggaagccgttgccgcgcgctatcgaagccgggcatttacattctatgatcctctgggggccgccgggtaccggcaaaacaactctcgctgaagtgattgcccgctatgcgaacgctgatgtggaacgtatttctgccgtcacctctggcgtgaaagagattcgcgaggcgatcgagcgcgcccggcaaaaccgcaatgcaggtcgccgcactattctttttgttgacgaagttcaccgtttcaacaaaagccagcaggatgcatttctgccacatattgaagacggcaccatcacttttattggcgcaaccactgaaaacccgtcgtttgagcttaattcggcactgctttcccgtgcccgtgtctatctgttgaaatccctgagtacagaggatattgagcaagtactaactcaggcgatggaagacaaaacccgtggctatggtggtcaggatattgttctgccagatgaaacacgacgcgccattgctgaactggtgaatggcgacgcgcgccgggcgttaaatacgctggaaatgatggcggatatggccgaagtcgatgatagcggtaagcgggtcctgaagcctgaattactgaccgaaatcgccggtgaacgtagcgcccgctttgataacaaaggcgatcgcttttacgatctgatttccgcactgcataagtcggtacgtggtagcgcacccgatgcggcgctgtactggtatgcgcgaattattaccgctggtggcgatccgttatatgtcgcgcgtcgctgtctggcgattgcgtctgaagacgtcggtaatgccgatccacgggcgatgcaggtggcaattgcggcctgggattgctttactcgcgttggcccggcggaaggtgaacgcgccattgctcaggcgattgtttacctggcctgcgcgccaaaaagcaacgctgtctacactgcgtttaaagccgcgctggccgatgctcgcgaacgcccggattatgacgtgccggttcatttgcgtaatgcgccgacgaaattaatgaaggaaatgggctacgggcaggaatatcgttacgctcatgatgaagcaaacgcttatgctgccggtgaggtttacttcccgccggaaatagcacaaacacgctattatttcccgacaaacaggggccttgaaggcaagattggcgaaaagctcgcctggctggctgaacaggatcaaaatagccccataaaacgctaccgttaa

SEQ ID NO: 33 - DNA, 1911 bp, Escherichia coli
gtgacggacgattttgcaccagacggtcagctggcgaaagcgataccaggctttaagccgcgagaaccacagcgacagatggcggtagccgtcacccaggcgatagaaaaaggccagccgctggtggtggaagcaggaaccggtacgggcaaaacctacgcttacctggctcctgcgctgcgggcgaaaaagaaagtcattatctcgaccggctcaaaagcgttgcaggatcagctctacagccgcgatttgccaacagtctcaaaggcattgaaatatacgggcaacgtggcgctgctgaaagggcgctcaaactacctctgcctcgaacgtctcgaacagcaggcgctggcggggggcgatctgccggtacaaatcttaagcgatgtgatcctgctgcgctcctggtctaatcaaacagtcgatggtgatatcagcacctgcgtcagcgtggcggaagattcacaggcgtggccgctggtcaccagcaccaacgacaactgtcttggcagcgactgcccgatgtataaagattgctttgtggtcaaagcacgtaaaaaagcgatggacgccgatgtggtggtggtaaaccatcatctctttctggcggatatggtggttaaagagagtggatttggcgaactgatcccggaagcggacgtcatgatcttcgacgaagcccaccagctaccggacattgccagccagtattttggtcagtcactctccagtcgacaactgctcgacctggcaaaagacatcaccatcgcctaccgcaccgaattaaaagacacccagcagttacaaaagtgcgctgatcgtcttgcccagagtgcgcaggattttcgtctgcaactcggtgagccaggttatcgcggtaacctgcgtgagctgttagctaatccgcaaattcagcgggcatttttactgctcgatgacaccctggaactttgttatgacgtggcgaaactgtcactggggcgttccgccttgctggatgcggcatttgagcgcgccacgttgtatcgcacacggctgaagcggctaaaagagatcaatcagccgggctacagctactggtacgaatgcacttcgcgccattttactctggctctcacgccgctcagcgtggcggataaattcaaagagttaatggcgcaaaaacccggtagctggatcttcacctcagcaacgctgtcggtgaacgacgatctgcatcatttcacctcgcggcttggcatcgaacaggccgagtcgttgctgttgcccagcccatttgattacagccgccaggcgttactctgtgtgctgcgcaatctgccgcaaaccaaccagccaggttctgctcgccagttagcggcaatgctgcgaccgatcatcgaagctaacaacggtcgttgttttatgctttgtacctcgcacgccatgatgcgcgatctggccgagcagttccgcgctaccatgacgcttcctgtattgttgcagggggaaaccagcaaagggcaactgttgcagcaatttgtcagcgccggtaatgcgcttcttgtggcaaccagcagtttctgggaaggggtggacgtgcgtggcgatacattgtcattggtaattatcgacaaattgccgtttacctcgccggatgatccactgttaaaagcgcgcatggaagattgtcgtttgcgcggtggcgacccgttcgatgaagtgcaactaccagatgccgtcattactctcaaacagggggtagggcgactgattcgcgacgccgacgatcgtggcgtgctggtgatttgtgacaatcggctggtgatgcgtccttacggcgcgacgtttctcgccagtctgccgcccgcgccacgcacccgtgacattgcccgtgcggttcgtttccttgcgataccatcctccaggtaa

SEQ ID NO: 34 - DNA, 414 bp, Escherichia coli
atggctaataaaccttcggcagaagaactgaaaaaaaatttgtccgagatgcagttttacgtgacgcagaatcatgggacagaaccgccatttacgggtcgtttactgcataacaagcgtgacggcgtatatcactgtttgatctgcgatgccccgctgtttcattcccaaaccaagtatgattccggctgtggctggcccagtttctacgaaccggtaagtgaagaatccattcgttatatcaaagacttgtcacatggaatgcagcgcatagaaattcgttgcggtaactgtgatgcccatctggggcatgtcttccccgacgggccgcagccaacgggcgaacgttattgtgttaactctgcctctttacgctttaccgatggcgaaaacggcgaagaaatcaacggttga

SEQ ID NO: 35 - DNA, 1968 bp, Escherichia coli
atgaccattgagaaaattttcaccccgcaggacgacgcgttttatgcggtgatcacccacgcggcggggccgcagggcgctctgccgctgaccccgcagatgctgatggaatctcccagcggcaacctgttcggcatgacgcagaacgccgggatgggctgggacgccaacaagctcaccggcaaagaggtgctgattatcggcactcagggcggcatccgcgccggagacggacgcccaatcgcgctgggctaccacaccgggcattgggagatcggcatgcagatgcaggcggcggcgaaggagatcacccgcaatggcgggatcccgttcgcggccttcgtcagcgatccgtgcgacgggcgctcgcagggcacgcacggtatgttcgattccctgccgtaccgcaacgacgcggcgatcgtgtttcgccgcctgatccgctccctgccgacgcggcgggcggtgatcggcgtagcgacctgcgataaagggctgcccgccaccatgattgcgctggccgcgatgcacgacctgccgactattctggtgccgggcggggcgacgctgccgccgaccgtcggggaagacgcgggcaaggtgcagaccatcggcgcgcgtttcgccaaccacgaactctccctgcaggaggccgccgaactgggctgtcgcgcctgcgcctcgccgggcggcgggtgtcagttcctcggcacggcgggcacctcgcaggtggtcgcggaggcgctgggtctggcgctgccgcactccgcgctggcgccgtccgggcaggcggtgtggctggagatcgcccgccagtcggcgcgcgcggtcagcgagctggatagccgcggcatcaccacgcgggatatcctctccgataaagccatcgaaaacgcgatggtgatccacgcggcgttcggcggctccaccaatttactgctgcacattccggccatcgcccacgcggcgggctgcacgatcccggacgttgagcactggacgcgcatcaaccgtaaagtgccgcgtctggtgagcgtgctgcccaacggcccggactatcacccgaccgtgcgcgccttcctcgcgggcggcgtgccggaggtgatgctccacctgcgcgacctcggcctgctgcatctggacgccatgaccgtgaccggccagacggtgggcgagaaccttgaatggtggcaggcgtccgagcgccgggcgcgcttccgccagtgcctgcgcgagcaggacggcgtagagccggatgacgtgatcctgccgccggagaaggcaaaagcgaaagggctgacctcgacggtctgcttcccgacgggcaacatcgctccggaaggttcggtgatcaaggccacggcgatcgacccgtcggtggtgggcgaagatggcgtataccaccacaccggccgggtgcgggtgtttgtctcggaagcgcaggcgatcaaggcgatcaagcgggaagagattgtgcagggcgatatcatggtggtgatcggcggcgggccgtccggcaccggcatggaagagacctaccagctcacctccgcgctaaagcatatctcgtggggcaagacggtgtcgctcatcaccgatgcgcgcttctcgggcgtgtcgacgggcgcctgcttcggccacgtgtcgccggaggcgctggcgggcgggccgattggcaagctgcgcgataacgacatcatcgagattgccgtggatcgtctgacgttaactggcagcgtgaacttcatcggcaccgcggacaacccgctgacgccggaagagggcgcgcgcgagctggcgcggcggcagacgcacccggacctgcacgcccacgactttttgccggacgacacccggctgtgggcggcactgcagtcggtgagcggcggcacctggaaaggctgtatttatgacaccgataaaattatcgaggtaattaacgccggtaaaaaagcgctcggaatttaa

SEQ ID NO: 36 - DNA, 717 bp, Escherichia coli
gtgggacgtaaatgggccaatattgttgctaaaaaaacggctaaagacggtgcaacgtctaaaatttatgcaaaattcggtgtagaaatctatgctgctgctaaacaaggtgaacccgatccagaattaaacacatctttaaaattcgttattgaacgtgcaaagcaggcacaagttccaaagcacgttattgataaagcaattgataaagccaaaggcggcggagatgaaacgttcgtgcagggacgttatgaaggctttggtcctaatggctcaatgattatcgccgagacattgacttcaaatgttaaccgtacgattgctaacgttcgcacaattttcaataaaaaaggcggcaatatcggagcggcaggttctgtcagctatatgtttgacaatacgggtgtgattgtatttaaagggacagaccctgaccatatttttgaaattttacttgaagctgaagttgatgttcgtgatgtgactgaagaagaaggtaacattgttatttatactgaacctactgaccttcataaaggaatcgcggctctaaaagcagctggaatcactgagttctcaacaacagaattagaaatgattgctcaatctgaagttgagctttccccagaagatttagaaatctttgaagggcttgttgatgcccttgaagatgacgacgatgtacaaaaagtttatcataacgtcgcaaatctctaa

SEQ ID NO: 37 - DNA, 258 bp, Escherichia coli
atggcgccgccactgtcgcctggctcgcgggtcctgatagccctcattcgggtctatcaacgcctgattagtccgctactcgggccgcattgtcgtttcactccaacctgttcaagctacggaattgaggcattgcgcaggtttggagtgataaaaggcagttggttgacggtgaaacgcgtattaaaatgccaccctttacaccctggtggtgacgatcccgtcccgcccggaccatttgataccagagaacactaa

SEQ ID NO: 38 - DNA, 1023 bp, Escherichia coli
atgaaaaaagtgttattgataatcttgttattgctggtggtactgggtatcgccgctggtgtgggcgtctggaaggttcgccatcttgccgacagcaaattgcttatcaaagaagagacgatatttaccctgaagccagggaccggacgtctggcgctcggtgaacagctttatgccgataagatcatcaatcgtccacgggtttttcaatggctgctgcgtatcgaaccggatctttctcactttaaagccgggacttaccgctttacaccgcagatgaccgtgcgcgagatgctgaaattgctggaaagcggtaaagaagcacagttccctctgcgactggtagaagggatgcgtctgagcgattacctcaagcaattgcgtgaggccccgtatatcaagcatacgctgagcgatgataagtacgccaccgtagcgcaggcacttgaactggaaaacccggagtggattgaaggttggttctggccagacacctggatgtataccgccaataccaccgatgtcgcgttactcaagcgagcgcacaagaaaatggtgaaagcggtcgatagcgcctgggaagggcgtgcggacggtctgccttataaagataaaaaccagttggtgacgatggcatcaattatcgaaaaagaaaccgccgttgccagtgaacgcgataaggttgcctcagtatttatcaaccgtttacgcattggtatgcgcctgcagaccgacccgaccgtgatttacgggatgggagagcgttataatggcaaactttctcgtgcagacctggaaacgccgacagcgtataacacctataccattaccggtctgccgccaggtgcgatagcgacgccgggggcggattcgctgaaggctgctgcgcatccggcaaaaacgccgtatctctattttgtggccgatggtaaaggtggtcacacgtttaataccaatcttgccagtcataacaagtctgtgcaggattatctgaaagtgcttaaggaaaaaaatgcgcagtaa

SEQ ID NO: 39 - DNA, 873 bp, Escherichia coli
atgctgcccgactcatcagtccgtttaaataaatacatcagcgaaagcggaatttgctcacgccgcgaagcggatcgctatatcgagcaaggcaatgtgttccttaatggcaagcgagccaccattggcgatcaggtgaaacccggcgacgttgtgaaagtaaacggtcagttgattgaacctcgggaagccgaagatttggtacttatcgccctgaacaagcccgttggtattgtaagcaccaccgaagatggcgagcgcgataacattgtcgatttcgttaaccacagcaaacgcgtgttcccgattggccgcctggataaagactcccaggggctgattttcctcaccaatcacggcgatctggtgaataagatcctgcgtgctggcaatgatcatgagaaagagtatctggtgacggtcgataaaccgattaccgaggagtttattcgcggcatgagtgcgggggtgccaatcctcgggacagtgaccaaaaagtgcaaagttaaaaaagaagcgccgtttgtcttccgcattaccctggtgcaggggctgaaccgtcagatccggcgcatgtgcgagcatttcggctatgaagtgaaaaagctggaacgcacgcgcatcatgaacgttagcttaagcggcattccgctgggggaatggcgcgatttaaccgacgatgagttaatcgacctctttaagctcattgaaaattcctcttccgaggtaaaacctaaagcgaaggccaaaccgaaaacagcgggcatcaaacgtccagtcgttaagatggaaaaaacggcggaaaaaggcggtcgcccggcgtccaacggtaagcgttttacctcgccggggcgtaaaaagaaggggcgctga

SEQ ID NO: 40 - protein, 159 aa, Escherichia coli
MRIGHGFDVHAFGGEGPIIIGGVRIPYEKGLLAHSDGDVALHALTDALLGAAALGDIGKLFPDTDPAFKGADSRELLREAWRRIQAKGYTLGNVDVTIIAQAPKMLPHIPQMRVFIAEDLGCHMDDVNVKATTTEKLGFTGRGEGIACEAVALLIKATK

SEQ ID NO: 41 - protein, 158 aa, Haemophilus influenzae
MIRIGHGFDVHAFGEDRPLIIGGVEVPYHTGFIAHSDGDVALHALTDAILGAAALGDIGKLFPDTDMQYKNADSRGLLREAFRQVQEKGYKIGNVDITIIAQAPKMRPHIDAMRAKIAEDLQCDIEQVNVKATTTEKLGFTGRQEGIACEAVALLIRQ

SEQ ID NO: 42 - protein, 158 aa, Bacillus subtilis
MFRIGQGFDVHQLVEGRPLIIGGIEIPYEKGLLGHSDADVLLHTVADACLGAVGEGDIGKHFPDTDPEFKDADSFKLLQHVWGIVKQKGYVLGNIDCTIIAQKPKMLPYIEDMRKRIAEGLEADVSQVNVKATTTEKLGFTGRAEGIAAQATVLIQKG

SEQ ID NO: 43 - protein, 161 aa, Synechocystis sp.
MTALRIGNGYDIHRLVGDRPLILGGVTIAHHLGLDGHSDADVLTHALMDALLGALSLGDIGHYFPPSDARWQGADSLKLLAQVHQLILERGWRINNLDNVIVAEQPKLKPHIQAMKENLAKVLTIDPDLIGIKATTNERLGPTGREEGIAAYSVALLIKEG

SEQ ID NO: 44 - protein, 399 aa, Treponema pallidum
MRRGGACVQKKEYLPLTSRQPGVCLLSEILVRALEARSFFLVVVTVPAGEVAYAESQVACDSRLSAFPSRTRPVILYVPGAHTRSASVRAGLDAMATHAPDVVLVHDGARPFVSVALIHSVLEATCRYGAAVPVIEATDTPKGVAADGSIETHLIRSRVRLAQTPQGFCYASLCAAHHRAATDGEQYTDDSELYARYGGTVHVCAGERSNVKITYPEDLEQRASEPALTRGISVLPCTEEGALRVGLGTDMHALCAGRPLILAGIHIPSKKGAQGHSDADVLAHASIDALLGAAGLGDIGTFFPSCDGRWKDAHSCALLRHTWQLVRAACWRLVNLDAVVCLEQPALHPFREAMRASLAQALDTHVTRVFVKAKTAERLGPVGSGAAVTAQVVVLLKKI

SEQ ID NO: 45 - protein, 406 aa, Helicobacter pylori
MSLIRVNGEAFKLSLESLEEDPFETKETLETLIKQTSVVLLAAGESKRFSRAIKKQWLRSHHTPLWLSVYESFKEALDFKEVILVVSELDYVYIQRHYPKIKLVKGGASRQESVRNALKVIDSTYTITSDVARGLANMEALKSLFLTLQQTSHYCIAPYLPCYDTAIYYNEALDREAIKLIQTPQLSHTKTLQSALNQGGFKDESSAILQAFPNSVSYIEGSKDLHKLTTSGDLKFFTPFFNPAKDTFIGMGFDTHAFIKDKPMVLGGVVLDCEFGLKAHSDGDALLHAVIDAILGAIKGGDIGEWFPDNDPKYKNASSKELLKIVLDFSQSIGFELLEMGATIFSEIPKITPYKPAILENLSQLLGLEKSQISLKATTMEKMGFIGKQEGLLVQAHVSMRYKQKL

(19) United States
(12) Patent Application Publication
(10) Pub. No.: US 2004/0086937 A1
(43) Pub. Date: May 6, 2004

(54) Novel method for identifying antibacterial compounds

(76) Inventors: Hannes Loferer, Geretsried (DE); Alexander Jacobi, Munchen (DE); Andrei Grigoriev, Germering (DE)

Correspondence Address: ROPES & GRAY LLP, ONE INTERNATIONAL PLACE, BOSTON, MA, 02110-2624, US

(21) Appl. No.: 09/973,674

(22) Filed: Oct. 9, 2001

Related U.S. Application Data
(63) Continuation-in-part of application No. PCT/EP00/03135, filed on Apr. 7, 2000.

(30) Foreign Application Priority Data
Apr. 9, 1999 (EP) EP 99107031.9
Feb. 4, 2000 (EP) EP 00102111.2

(51) Int. Cl. G01N 33/53

(52) U.S. Cl. 435/7.1

(57) ABSTRACT

The present invention relates to a method for identifying an antagonist or inhibitor of the expression of a gene encoding a polypeptide essential for bacterial growth or survival as well as for an antagonist or inhibitor of said polypeptide. The invention further relates to a method for improved antagonists or inhibitors. The invention also provides an antagonist or inhibitor of the activity of said polypeptide. The invention is further related to a method for producing a therapeutic agent in a composition comprising said antagonist or inhibitor. Furthermore, the invention is related to the use of the polypeptide and the antagonist or inhibitor as well as to a method to identify a surrogate marker.

### REFERENCE TO RELATED APPLICATIONS

This application is a continuation-in-part application claiming priority to international application number PCT/EP00/03135, filed on Apr. 7, 2000 and designated the U.S., which claims priority to EP 99107031.9, filed on Apr. 9, 1999 and EP 00102111.2, filed on Feb. 4, 2000, the entire contents of which are incorporated by reference herein.

### BACKGROUND OF THE INVENTION

The present invention relates to a method for identifying an antagonist or inhibitor of the expression of a gene encoding a polypeptide essential for bacterial growth or survival as well as for an antagonist or inhibitor of said polypeptide. The invention further relates to a method for improved antagonists or inhibitors. The invention also provides an antagonist or inhibitor of the activity of said polypeptide. The invention is further related to a method for producing a composition comprising said antagonist or inhibitor. Furthermore, the invention is related to the use of the polypeptide and the antagonist or inhibitor as well as to a method to identify a surrogate marker.

Several documents are cited throughout the text of this specification. Each of the documents cited herein (including any manufacturer's specifications, instructions, etc.) are hereby incorporated by reference; however, there is no admission that any document cited is indeed prior art of the present invention.

Since the beginning of the 1980s, a new trend has been observed in the industrialized countries. On the one hand, resistances to antibiotics have increased, which make it difficult or even impossible to treat many of the disease-causing agents. On the other hand, new infectious diseases, which had been unknown up to now, arise, and old diseases return. For example, diphtheria and tuberculosis are old epidemics and increasingly surmounting in many different parts of the world. Especially tuberculosis (TB), a chronic infectious disease that is generally caused by infection with Mycobacterium tuberculosis, is a disease of major concern. Each year, 8 to 10 million new cases of TB are described, and, causing more than three million deaths per year, TB is a major disease in developing countries as well as an increasing problem in developed areas of the world due to, for example, antibiotic resistance.

Additionally, M. bovis BCG vaccination has failed to protect against TB in several trials (WHO, Tech. Rep. Ser. (1980), 651, 1-15) for reasons that are not entirely clear (Fine, Tubercle 65 (1984), 137-153). It has been shown that the vaccine strain of M. bovis BCG only confers protection against the severe form of miliary tuberculosis in children (Fine, Lancet 346 (1995), 1339-1345). In contrast, its protective capacity against the most common form, pulmonary tuberculosis in adults, is low and highly variable (Colditz (1994), JAMA 271, 698).

The causes for this new trend are complex: mainly, the increasing number of antibiotic applications in medicine and agriculture often combined with an improper and uncontrolled use, helps to establish resistant organisms and generate the threat of bacterial infections resistant to all available therapies.

Conventional techniques of developing antibiotics, i.e. synthesis of candidate substances and screening for antibacterial substances, even though speeded up by several orders of magnitude by the use of combinatorial approaches in recent years (e.g. U.S. Pat. No. 5,324,483, U.S. Pat. No. 5,545,568), are still too inefficient as they involve multiple screening steps of hundreds or thousands of more or less randomly chosen substances for efficiency in combating various infectious agents.

Therefore, it is a major concern to fight the growing number of bacterial infections due to an increased frequency of multiple antibiotic resistances and to improve the available antibacterial therapies.

Thus, the technical problem underlying the present invention was to provide a method and means for the development of an additional effective antibacterial therapy of infected humans and animals that can be used for the treatment of a broad spectrum of bacterial infections or diseases or disorders related to bacterial infections. The solution to this technical problem is achieved by providing the embodiments characterized in the claims.

Accordingly, the present invention relates to a method for identifying an antagonist or inhibitor of the expression of a gene encoding a polypeptide essential for bacterial growth wherein said gene is selected from the group consisting of ygbB, yfhC, yacE, ychB, yejD, yrfl, yggJ, yjeE, yiaO, yrdC, yhbC, ygbP, ybeY, gcpE, kdtB, pfs, ycaj, b1808, yeaA, yagF, b1983, yidD, yceG and/or yjbC the sequence of said genes being shown in FIG. 1, or a fragment or derivative or ortholog thereof, said method comprising the steps of

(a) testing a candidate antagonist or inhibitor or a sample comprising a plurality of said candidate antagonists or inhibitors for the inhibition or reduction of transcription of said gene or a fragment or derivative thereof, or

(b) testing a candidate antagonist or inhibitor or a sample comprising a plurality of said candidate antagonists or inhibitors for the inhibition or reduction of translation of mRNA transcribed from said gene or a fragment or derivative thereof; and

(c) identifying an antagonist or inhibitor or a sample comprising a plurality of said candidate antagonists or inhibitors that tests positive in step (a) and/or (b).

The term “antagonist” or “inhibitor” as used herein means naturally occurring and synthetic compounds capable of counteracting or inhibiting an activity of a gene or gene product or interactions of the gene or gene product with other genes or gene products. Determining whether a compound is capable of inhibiting or counteracting specific gene expression can be done, for example, by Northern blot analysis, Western blot analysis or proteome analysis. It can further be done by monitoring the phenotypic characteristics of a bacterial cell contacted with the compounds and compare it to that of a wild-type cell. In an additional embodiment, said characteristics may be compared to that of a cell contacted with a compound which is either known to be capable or incapable of suppressing or activating the protein or gene, respectively, according to the invention. For example, the bacterial cell can be a transgenic cell and the phenotypic characteristics comprises a readout system. Further examples of determining whether a compound is capable of inhibiting or counteracting specific gene expression are described below.

The term “expression” means the production of a protein or nucleotide sequence in a cell. However, said term also includes expression of the protein in a cell-free system. It includes transcription into an RNA product, and/or translation into a polypeptide from a DNA encoding that product.

The term “transcription” as used herein means a DNA template dependent synthesis of a ribonucleic acid polymer encoding a polypeptide or a regulatory sequence. The term “translation” as used herein means the polymerization of a polypeptide that is encoded by an RNA molecule by a protein complex.

As used in accordance with the present invention, the term “fragment or derivative” denotes any variant the amino acid or nucleotide sequence of which deviates in its primary structure, e.g., in sequence composition or in length as well as to analogue components. For example, one or more amino acids of a polypeptide may be replaced in said fragment or derivative as long as the modified polypeptides remain functionally equivalent to their described counterparts. The term “fragment or derivative” further denotes compounds analog to an antagonist or inhibitor that should have a stabilized electronic configuration and molecular conformation that allows key functional groups to be presented to the mentioned polypeptide in substantially the same way as the antagonist and inhibitor. The variant of the polypeptide may be a naturally occurring allelic variant of the polypeptide or non-naturally occurring variants of those polynucleotides.

The term “orthologs” as used herein means homologous sequences in different species that evolved from a common ancestoral gene by speciation. Normally, orthologs retain the same function in the course of evolution. However, orthologous genes may or may not be responsible for a similar function (see, e.g., the glossary of the “Trends Guide to Bioinformatics”, Trends Supplement 1998, Elsevier Science). Orthologous genes, nucleic acids or proteins comprise genes, nucleic acids or proteins which have one or more sequences or structural motifs in common. For example, the sequence motifs of proteins can comprise short, i.e. repetitive sequences or amino acid positions conserved in the primary structure and/or conserved in higher protein structures, e.g. secondary or tertiary structure. Orthologous nucleic acids or genes can comprise molecules having short stretches of one or more homologous (same or similar) sequences, for example protein binding boxes or structure forming boxes. Methods for the identification of a candidate ortholog of a gene or polypeptide described herein are known to those skilled in the art and are described for example in Sambrook et al. (1989), Molecular Cloning: A Laboratory Manual, Cold Spring Harbor Laboratory Press, New York, or Ausubel (1994), Current Protocols in Mol. Biol. The person skilled in the art knows how to identify orthologous genes, nucleic acids or polypeptides by computer supported analysis (e.g. BLAST) of known sequences and its interpretation.

The terms “gene”, “polynucleotide”, “nucleic acid sequence”, “nucleotide sequence”, “DNA sequence” or “nucleic acid molecule” as used herein refer to polymeric forms of nucleotides of any length, either ribonucleotides or deoxyribonucleotides and only to the primary structure of the molecule. Thus, these terms include double- and single-stranded DNA, and RNA. They also include known types of modifications, for example, methylation, “caps” substitution of one or more of the naturally occurring nucleotides with an analog. Preferably, the DNA sequence of the invention comprises a coding sequence encoding at least the mature form of the above defined protein, i.e. the protein which is posttranslationally processed in its biologically active form, for example due to cleavage of leader or secretory sequences or a proprotein sequence or other natural proteolytic cleavage points.

The term “plurality of candidate antagonists or inhibitors” is to be understood as a plurality of substances which may or may not be identical.

Said antagonists or inhibitors or plurality of candidate antagonists or inhibitors may be chemically synthesized or microbiologically produced and/or comprised in, for example, samples, e.g., cell extracts from, e.g., plants, animals or microorganisms. Furthermore, said compound(s) may be known in the art but hitherto not known to be capable of suppressing or inhibiting said polypeptide. The reaction mixture may be a cell free extract or may comprise a cell or tissue culture. Suitable set ups for the method of the invention are known to the person skilled in the art and are, for example, generally described in Alberts et al., Molecular Biology of the Cell, third edition (1994), in particular Chapter 17. The plurality of compounds may be, e.g., added to the reaction mixture, culture medium, injected into the cell or sprayed onto the plant.

By combining computational processing of genomic information with microbial genetics, the inventors have been able to identify 24 E. coli essential genes and their respective orthologs (FIG. 3) that fulfill several criteria for being attractive antibacterial targets: hypothetical open reading frames, coding for essential functions (mutation is lethal for growth in rich media), broad conservation (orthologs are present in a wide range of bacteria including H. influenza, S. pneumoniae, H. pylori, and B. burgdorferi) (FIG. 3) and low toxicity potential in higher organisms (mostly no orthologs are identified in the simple eukaryote S. cerevisiae). Thus, an antagonist or inhibitor of the expression of such an essential gene or of its function provides the key for an antibacterial therapy. The inventors assume that said antagonist or inhibitor stops or reduces bacterial growth and/or mediates bacterial death.

Thus, the method of the present invention provides the options of development of new broad spectrum antibiotics against new pharmaceutical important targets. The findings of the present invention are particularly important in view of the drawbacks of the present forms of treatment of bacterial infections, diseases and disorders related to bacterial infections.

In line with the above, the present invention also relates to a method for testing a candidate antagonist or inhibitor of a polypeptide or mRNA essential for bacterial growth or survival encoded by a gene selected from the group consisting of ygbB, yfhC, yacE, ychB, yejD, yrfl, yggJ, yjeE, yiaO, yrdC, yhbC, ygbP, ybeY, gcpe, kdtB, pfs, ycaj, b1808, yeaA, yagF, b1983, yidD, yceG and/or yjbC or a fragment, derivative or ortholog thereof comprising the steps of:

(a) contacting a bacterial cell with candidate antagonist or inhibitor or a sample comprising a plurality of said candidate antagonists or inhibitors; and

(b) determining whether said contacting leads to cell growth inhibition and/or cell death resulting from eliminating the function of at least one of said genes.

In a further embodiment, the present invention relates to a method for testing a candidate antagonist or inhibitor of the function of a gene essential for bacterial growth or survival wherein said gene is selected from the group consisting of ygbB, yfhC, yacE, ychB, yejD, yrfl, yggJ, jeE, yiaO, yrdC, yhbC, ygbP, ybey, gcpE, kdtB, pfs, ycaJ, b1808, yeaA, yagF, b1983, yidD, yceG and/or yjbC or a fragment, derivative or ortholog thereof, comprising the steps of:

(a) contacting a bacterial cell comprising said gene with a candidate antagonist or inhibitor or a sample comprising a plurality of said candidate antagonists or inhibitors; and

(b) determining whether said contacting leads to cell growth inhibition and/or cell death resulting from eliminating the function of at least one of said genes.

In the context of these embodiments, “testing whether said contacting leads to gene-specific cell growth inhibition and/or cell death” means testing for cell growth inhibition and/or cell death that is mediated and/or caused through an antagonistic or inhibitory influence of a candidate antagonist or inhibitor on a gene selected from the group consisting of ygbB, yfhC, yacE, ychB, yejD, yrfl, yggj, yjee, yiaO, yrdC, yhbC, ygbP, ybeY, gcpE, kdtB, pfs, ycaj, b1808, yeaA, yagF, b1983, yidD, yceG and/or yjbC or a fragment, derivative or ortholog thereof.

In searching for inhibitors or antagonists effecting growth inhibition or cell killing through a gene-specific action in a large number of candidate inhibitor or antagonist molecules, one will usually first screen for non-gene-specific growth inhibition or cell killing, followed by further experiments to confirm the action of an inhibitor or antagonist through interaction with a specific gene or one of its gene products, i.e. mRNA or protein.

A screen for compounds which non-gene-specifically inhibit growth of or kill cells will usually consist of the incubation of a large number of candidate compounds with such cells under conditions that, absent a growth inhibitory or cell killing effect of a compound, allow the persistence and growth of said cells. Where the contact with a candidate compound results in impairment of such growth, a candidate inhibitor or antagonist is isolated.

Bacteria, for which was shown that a gene as mentioned above expressed is essential, can be used in such a proliferation assay to identify both ligands and potential antagonists or inhibitors to said polypeptide encoded by said essential gene. For example, E. coli are grown in culture medium and incorporation of DNA precursors such as 3H-thymidine or 5-bromo-2′-deoxyuridine (BrdU) is monitored as a parameter for DNA synthesis and cellular proliferation. Cells which have incorporated BrdU into DNA can be detected using a monoclonal antibody against BrdU and measured by an enzyme or fluorochrome-conjugated second antibody. The reaction is quantitated by fluorimetry or by spectrophotometry. The ability of the compound to be screened to inhibit proliferation may then be quantified. Further methods to determine growth and proliferation of bacteria are well known in the art, for example in Drews, Mikrobiol. Praktikum, Berlin, 1976.

To test whether a candidate antagonist or inhibitor or a plurality thereof acts “gene-specifically” can be investigated by a number of different methods, e.g.:

A) Gene Specific Change/Shift of Susceptibility to Said Antagonists/Inhibitors As Read Out, e.g. by:

1) Over-expression rescue. The putative target gene is cloned into a suitable vector which contains a regulatable promotor thereby enabling the investigator to “switch on” or “switch off” target gene over-expression. (Escherichia coli and Salmonella 2nd ed., Neidhardt F C, ASM Press, 1996, p. 2269; McMurry L M, Oethinger M, Levy S B (1998). Triclosan targets lipid synthesis. Nature 394: 531-532; Leszczynska K, Bolhuis A, Leenhouts K, Venema G, Ceglowski P (1995). Cloning and Molecular Analysis of the Dihydrofolate Reductase Gene from Lactococcus lactis. Appl Env Microbiol 61: 561-566). If over-expression of the putative target gene results in at least partial rescue of the phenotype caused by the candidate antagonist/inhibitor, then the phenotype (cell death/growth inhibition, etc.) is at least partially caused by gene-specific inhibition of one or more putative target genes. No specific function of the putative target gene is required for this type of testing.

2) Sensitizing the genetic background by Limited expression. The expression level of the putative target gene can be lowered by:

a) use of conditional lethal mutants, e.g. temperature-sensitive mutants or promoter-regulated target gene expression (Huang B, Rifkin MR, Luck DJ, 1977, Temperature-sensitive mutations affecting flagellar assembly and function in Chlamydomonas reinhardtii. J Cell Biol 72: 67-85; Smuda J W, Carter B J, 1991, Adeno-associated viruses having nonsense mutations in the capsid genes: growth in mammalian cells containing an inducible amber suppressor. Virology 184: 310-318), or

b) regulated expression of a gene specific antisense RNA. (Biochemistry 2nd ed., Voet D, Wiley & Sons, 1995, p. 1007).

In both types of experiments the expression of the putative target gene at the protein level is down-regulated. The sensitivity of such cells to the candidate inhibitor or antagonist is then determined. If cells show a sensitivity towards the candidate inhibitor or antagonist roughly scaling with the expression level of the putative target gene, this may again be taken as an indicator for a specific action of the candidate inhibitor or antagonist on the target gene. Again, specific biochemical function of a gene is not required for antisense-regulated gene expression.

3) Changing the biochemical stability of target gene products. The half-life of a gene specific product, i.e. mRNA or protein, may be lowered or increased, e.g. by genetically engineering the stability of the mRNA or protein encoded by the target gene by introduction of stabilizing mRNA secondary structures or protein stabilizing amino acid residues, or introduction of RNase cleavage or protease cleavage sites/protease targeting signals. (Biochemistry 2nd ed., Voet D, Wiley & Sons, 1995, pp. 1010, 1167; Escherichia coli and Salmonella 2nd ed., Neidhardt F C, ASM Press, 1996, pp. 850). If cells show a sensitivity towards the candidate inhibitor or antagonist roughly scaling with the steady state level of the putative target gene product, this may again be taken as an indicator for a specific action of the candidate inhibitor or antagonist on the target gene.

In an experiment according to the above mechanisms, it may be investigated whether the growth inhibitory or cell killing effect of a candidate inhibitor or antagonist can be reversed by raising or enhanced by lowering the amount of mRNA/protein encoded by the putative target gene present in the cell. An observation of such a dependence of the sensitivity of cells on the amount of mRNA/protein present is a good indicator for a specific activity of the candidate inhibitor or antagonist on the target gene.

4) However, some of the above methods may require detailed knowledge about the putative target gene. If only limited knowledge of the target gene is available, slightly different methods may be employed. For example, if:

a) the pathway the target gene is involved is known but not the enzymatic activity: particular enzymes from an alternative pathway can rescue the destroyed cellular function thereby proving specificity of the candidate antagonist or inhibitor. (Campos N, Rodriguez-Concepcion M, Sauret-Gueto S, Gallego F, Lois L-M, Boronat A, 2001, Escherichia coli engineered to synthesize isopentenyl diphosphate and dimethylallyl diphosphate from mevalonate: a novel system for the genetic analysis of the 2-C-methyl-D-erythritol 4-phosphate pathway for isoprenoid biosynthesis. Biochem. J. 353: 59-67).

b) the product of the enzymatic reaction is known but neither substrate nor co-factors is known: if chemically synthesized product can rescue the destroyed cellular function, then the specificity of the candidate antagonist or inhibitor against the candidate target gene is established. (Escherichia coli and Salmonella 2nd ed., Neidhardt F C, ASM Press, 1996, pp. 580).

c) substrate of the enzymatic reaction is known but neither product nor co-factors is known: if feeding cells with chemically synthesized or genetically overproduced substrate can enhance the turnover rate of the putative target gene thereby rescuing the inhibited or antagonized cellular function, a proof of specificity of the candidate antagonist or inhibitor for the target gene can be established. (Escherichia coli and Salmonella 2nd ed., Neidhardt FC, ASM Press, 1996, pp. 580).

B) Investigation of Direct Interaction, e.g. by:

5) Three-hybrid-technology: the putative target gene is fused to, for example, an activation domain (AD, e.g. GAL4 activation domain) of a known gene, and a gene encoding a protein for which a tight-binding ligand is known (e.g. dihydrofolate reductase DHFR, binds trimethoprim and methotrexate with high affinity and specificity) is fused to a DNA binding domain (BD, e.g. GAL4 DNA binding domain). The resulting chimeric translation products of DHFR and the target gene are brought into contact with a candidate antagonist or inhibitor molecule chemically linked to trimethoprim or methotrexate in an environment containing a reporter gene under the control of a promoter comprising the binding motif of the BD (e.g. GAL-UAS-O-gal). Provided, the candidate antagonist or inhibitor molecule binds to the putative target gene with high affinity and specificity, the respective activation and DNA binding domains are brought into close proximity via the bridging interaction of the (candidate inhibitor or antagonist/trimethoprim or methotrexate) hybrid molecule, initiating transcription activation of the reporter gene (Drees B L, 1999, Progress and variations in two-hybrid and three-hybrid technologies. Curr Opin Chem Biol 3: 64-70; WO 9741255). A direct binding between the candidate target gene product and the inhibitor/antagonist is a strong indication that the phenotype caused by the inhibitor/antagonist is through inhibiting/antagonizing the function of the candidate target gene product.

6) Biochemical determination of binding: The binding of purified target protein to candidate antagonist or inhibitor may be determined directly by, e.g., surface plasmon resonance or other spectroscopical means (Nelson R W, Nedelkov D, Tubbs K A, 2000, Biosensor chip mass spectrometry: a chip-based proteomics approach. Electrophoresis 21: 1155-63). A high affinity and specificity of the candidate inhibitor or antagonist is a good indicator for its effecting the growth inhibition/cell killing observed in a non-gene-specific growth inhibition/cell killing assay through action on the target gene.

Preferably, the antagonist or inhibitor binds to the gene product, i.e. the RNA or polypeptide, specifically encoded by said gene.

For example, a candidate antagonist or inhibitor not known to be capable of binding to an polypeptide encoded by a essential gene as described above can be tested to bind thereto comprising contacting a bacterial cell comprising an isolated molecule encoding said polypeptide with a candidate antagonist or inhibitor under conditions permitting binding of ligands known to bind thereto, detecting the presence of any bound ligand, and thereby determining whether such candidate antagonist or inhibitor inhibits the binding of a ligand to a polypeptide as described above.

Proteins that bind to a polypeptide as described above and might inhibit or counteract to said polypeptide can be “captured” using the yeast two-hybrid system (Fields, Nature 340 (1989), 245-246). A modified version of the yeast two-hybrid system has been described by Roger Brent and his colleagues (Gyuris, Cell 75 (1993), 791-803; Zervos, Cell 72 (1993), 223-232). Briefly, a domain of the polypeptide is used as bait for binding compounds. Positives are then selected by their ability to grow on plates lacking leucine, and then further tested for their ability to turn blue on plates with X-gal, as previously described in great detail (Gyuris, supra; WO 95/31544). Once amino acid sequences are identified which bind to a polypeptide essential for bacterial growth or survival, these sequences can be screened for antagonist activity using, for example, the proliferation assay described above or used for screening for antagonists of said binding.

Another assay which can be performed to identify inhibitors and antagonists involves the use of combinatorial chemistry to produce random peptides which then can be screened for both binding affinity and antagonist effects. One such assay has recently been performed using random peptides expressed on the surface of a bacteriophage (Wu (1996), Nature Biotechnology 14, 429-431).

In a preferred embodiment of the method of the present invention said method further comprises identifying an antagonist or inhibitor optionally from said sample of candidate antagonists or inhibitors.

If a sample contains a candidate antagonist or inhibitor, or a plurality of candidate antagonists or inhibitors, as identified in the method of the invention, then it is either possible to isolate the candidate antagonists or inhibitors from the original sample identified as containing the compound capable of suppressing or inhibiting bacterial growth or survival, or one can further subdivide the original sample, for example, if it consists of a plurality of different candidate antagonists or inhibitors, so as to reduce the number of different substances per sample and repeat the method with the subdivisions of the original sample. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. As regards the identification of candidate antagonists or inhibitors by any of the above-referenced embodiments of the invention, a variety of formats or tools is available to the person skilled in the art. Thus, several methods are known to the person skilled in the art for producing and screening large libraries to identify compounds having specific affinity for a target. These methods include the phage-display method in which randomized peptides are displayed from phage and screened by affinity chromatography to an immobilized receptor; see, e.g., WO 91/17271, WO 92/01047, U.S. Pat. No. 5,223,409. In another approach, combinatorial libraries of polymers immobilized on a chip are synthesized using photolithography; see, e.g., U.S. Pat. No. 5,143,854, WO 90/15070 and WO 92/10092. The immobilized polymers are contacted with a labeled receptor and scanned for label to identify polymers binding to the receptor. The synthesis and screening of peptide libraries on continuous cellulose membrane supports that can be used for identifying binding ligands of the polypeptide of the invention and thus possible inhibitors and antagonists is described, for example, in Kramer, Methods Mol. Biol. 87 (1998), 25-39. This method can also be used, for example, for determining the binding sites and the recognition motifs in the polypeptide as described above. In like manner, the substrate specificity of the DnaK chaperon was determined and the contact sites between human interleukin-6 and its receptor; see Rüdiger, EMBO J. 16 (1997), 1501-1507 and Weiergraber, FEBS Lett. 379 (1996), 122-126, respectively.

Furthermore, the above-mentioned methods can be used for the construction of binding supertopes derived from the polypeptide of the invention. A similar approach was successfully described for peptide antigens of the anti-p24 (HIV-1) monoclonal antibody; see Kramer, Cell 91 (1997), 799-809. A general route to fingerprint analyses of peptide-antibody interactions using the clustered amino acid peptide library was described in Kramer, Mol. Immunol. 32 (1995), 459-465. In addition, antagonists or inhibitors of a polypeptide described above can be derived and identified from monoclonal antibodies that specifically react with said polypeptide in accordance with the methods as described in Doring, Mol. Immunol. 31 (1994), 1059-1067.

More recently, WO 98/25146 described further methods for screening libraries of complexes for compounds having a desired property, especially, the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with the proteins according to the invention or nucleic acid molecules encoding such molecules are, for example, the in vitro screening with the phage display system as well as filter binding assays or “real time” measuring of interaction using, for example, the BIAcore apparatus (Pharmacia).

All these methods can be used in accordance with the present invention to identify antagonists and inhibitors of the polypeptide of the invention.

Additionally, the present invention relates in a preferred embodiment to a method comprising improving inhibitors or antagonists identified by peptidomimetics or by applying phage display or combinatorial library technique step(s). Peptidomimentics, phage display and combinatorial library techniques are well-known in the art and can be applied by the person skilled in the art without further ado to the improvement of the antagonist or inhibitor that is identified by the basic method referred to herein above.

Methods for the generation and use of peptidomimetic combinatorial libraries are described in the prior art, for example in Ostresh, Methods In Enzymology 267 (1996), 220-236; Dosner, Bioorg. Med. Chem. 4 (1996), 709-715; Beeley, Trends Biotechn. 12 (1994), 213-216; al-Obeidi, Mol. Biotechn. 9 (1998), 205-223; Wiley, Med. Res. Rev. 13 (1993), 327-384; Bohm, J. Comput. Aided Mol. Des. 10 (1996), 265-272; and Hruby, Biopolymers 43 (1997), 219-266.

Various sources for the basic structure of such an antagonist or inhibitor can be employed and comprise, for example, mimetic analogs of the polypeptide of the invention. Mimetic analogs of the polypeptide of the invention or biologically active fragments thereof can be generated by, for example, substituting the amino acids that are expected to be essential for the biological activity with, e.g., stereoisomers, i.e. D-amino acids; see e.g., Tsukida, J. Med. Chem. 40 (1997), 35343541. Furthermore, in case fragments are used for the design of biologically active analogs promimetic components can be incorporated into a peptide to reestablish at least some of the conformational properties that may have been lost upon removal of part of the original polypeptide; see, e.g., Nachman, Regul. Pept. 57 (1995), 359-370. Furthermore, the polypeptide can be used to identify synthetic chemical peptide mimetics that bind to or can function as a ligand, substrate, binding partner or the receptor of the polypeptide as effectively as does the natural polypeptide; see, e.g., Engleman, J. Clin. Invest. 99 (1997), 2284-2292.

The structure-based design and synthesis of low-molecular-weight synthetic molecules that mimic the activity of the native biological polypeptide is further described in, e.g., Dowd, Nature Biotechnol. 16 (1998), 190-195; Kieber-Emmons, Current Opinion Biotechnol. 8 (1997), 435441; Moore, Proc. West Pharmacol. Soc. 40 (1997), 115-119; Mathews, Proc. West Pharmacol. Soc. 40 (1997), 121-125; Mukhija, European J. Biochem. 254 (1998), 433-438.

It is also well known to the person skilled in the art, that it is possible to design, synthesize and evaluate mimetics of small organic compounds that, for example, can act as a substrate or ligand to a polypeptide as encoded by the essential gene as identified above. For example, it has been described that D-glucose mimetics of hapalosin exhibited similar efficiency as hapalosin in antagonizing multidrug resistance assistance-associated protein in cytotoxicity; see Dinh, J. Med. Chem. 41 (1998), 981-987.

The essential gene described above or the RNA encoded thereof, as has been described above, can also serve as a target for antagonists or inhibitors. Antagonists may comprise, for example, proteins that bind to the mRNA of said gene, thereby destabilizing the native conformation of the mRNA and disturbing transcription and/or translation. Furthermore, methods are described in the literature for identifying nucleic acid molecules such as an RNA fragment that mimics the structure of a defined or undefined target RNA molecule to which a compound binds inside of a cell resulting in retardation of cell growth or cell death; see, e.g., WO 98/18947 and references cited therein. These nucleic acid molecules can be used for identifying unknown compounds of pharmaceutical and/or agricultural interest, and for identifying unknown RNA targets for use in treating a disease. These methods and compositions can be used in screening for novel antibiotics, bacteriostatics, or modifications thereof or for identifying compounds useful to alter expression levels of proteins encoded by a nucleic acid molecule. Alternatively, for example, the conformational structure of the RNA fragment which mimics the binding site can be employed in rational drug design to modify known antibiotics to make them bind more avidly to the target. One such methodology is nuclear magnetic resonance (NMR), which is useful to identify drug and RNA conformational structures. Still other methods are, for example, the drug design methods as described in WO 95/35367, U.S. Pat. No. 5,322,933, where the crystal structure of the RNA fragment can be deduced and computer programs are utilized to design novel binding compounds which can act as antibiotics.

The candidate antagonists and inhibitors which can be tested and identified according to a method of the invention may be taken from expression libraries, e.g., cDNA expression libraries, peptides, proteins, nucleic acids, antibodies, small organic compounds, hormones, peptidomimetics, PNAs or the like (Milner, Nature Medicine 1 (1995), 879-880; Hupp, Cell 83 (1995), 237-245; Gibbs, Cell 79 (1994), 193-198 and references cited supra). Furthermore, genes encoding a putative regulator of an essential bacterial protein and/or which exert their effects up-or downstream said protein may be identified using, for example, insertion mutagenesis using, for example, gene targeting vectors known in the art (see, e.g., Hayashi, Science 258 (1992), 1350-1353; Fritze and Walden, Gene activation by T-DNA tagging. In Methods in Molecular biology 44 (Gartland, K. M. A. and Davey, M. R., eds). Totowa: Human Press (1995), 281-294) or transposon tagging (Chandlee, Physiologia Plantarum 78 (1990), 105-115). Said compounds can also be functional derivatives or analogues of known inhibitors or antagonists. Such useful compounds can be for example transacting factors which bind an above-described polypeptide.

Identification of transacting factors can be carried out using standard methods in the art (see, e.g., Sambrook, supra, and Ausubel, supra). To determine whether a protein binds to the protein or regulatory sequence of the invention, standard native gel-shift analyses can be carried out. In order to identify a transacting factor which binds to the protein or regulatory sequence of the invention, the protein or regulatory sequence of the invention can be used as an affinity reagent in standard protein purification methods, or as a probe for screening an expression library. The identification of nucleic acid molecules which encode proteins which interact with the polypeptide described above can also be achieved, for example, as described in Scofield (Science 274 (1996), 2063-2065) by use of the so-called yeast “two-hybrid system”; see also the appended example. In this system, e.g., the protein encoded by the nucleic acid molecules identified in this invention or a smaller part thereof is linked to the DNA-binding domain of the GAL4 transcription factor. A yeast strain expressing this fusion gene and comprising a lacZ reporter gene driven by an appropriate promoter, which is recognized by the GAL4 or LexA transcription factor, is transformed with a library of cDNAs which will express plant genes or fragments thereof fused to an activation domain. Thus, if a peptide encoded by one of the cDNAs is able to interact with the fusion peptide comprising a peptide of a protein of the invention, the complex is able to direct expression of the reporter gene. In this way the nucleic acid molecules and the encoded peptide can be used to identify peptides and proteins interacting with the polypeptide described above. It is apparent to the person skilled in the art that this and similar systems may then further be exploited for the identification of inhibitors or antagonists of the polypeptide.

Once the transacting factor is identified, modulation of its binding to or regulation of expression of the polypeptide described above can be pursued, beginning with, for example, screening for inhibitors against the binding of the transacting factor to the protein specified in accordance with the present invention. Inhibition of bacterial growth could then be achieved by applying the transacting factor (or its inhibitor). In addition, if the active form of the transacting factor is a dimer, dominant-negative mutants of the transacting factor could be made in order to inhibit its activity.

Thus, the present invention also relates to the use of the polypeptide as defined above for the identification of antagonists or inhibitors of a polypeptide essential for bacterial growth or survival.

In another embodiment, the present invention relates to a method for designing an improved antagonist or inhibitor for the treatment of a bacterial infection or disorder or disease related to a bacterial infection comprising the steps of

(a) identification of the binding site of an antagonist or inhibitor to the polypeptide ygbB, yfhC, yacE, ychB, yejD, yrfl, yggJ, yjeE, yiaO, yrdC, yhbC, ygbP, ybeY, gcpE, kdtB, pfs, ycaj, b1808, yeaA, yagF, b1983, yidD, yceG and/or yjbC, the sequence of said genes being shown in FIG. 1, or identified according to the method of the present invention, by site-directed mutagenesis and chimeric polypeptide studies;

(b) molecular modeling of both the binding site of said antagonist or inhibitor and the structure of said polypeptide; and

(c) modification of said antagonist or inhibitor to improve its binding specificity or affinity for the polypeptide.

Biological assays as described above or other assays such as assays based on crystallography or NMR may be employed to assess the specificity or potency of the antagonist or inhibitor wherein the decrease of one or more activities of the polypeptide may be used to monitor said specificity or potency. All techniques employed in the various steps of the method of the invention are conventional or can be derived by the person skilled in the art from conventional techniques without further ado.

For example, identification of the binding site of said antagonist or inhibitor by site-directed mutagenesis and chimerical protein studies can be achieved by modifications in the (poly)peptide primary sequence that affect the antagonist's or inhibitor's affinity; this usually allows to precisely map the binding pocket for the drug. Identification of binding sites may be assisted by computer programs. Thus, appropriate computer programs can be used for the identification of interactive sites of a putative antagonist or inhibitor and the polypeptide of the invention by computer assisted searches for complementary structural motifs (Fassina, Immunomethods 5 (1994), 114-120).

As regards step (b), the following protocols may be envisaged: Once the effector site for antagonists or inhibitors has been mapped, the precise residues interacting with different parts of the antagonists or inhibitors can be identified by combination of the information obtained from mutagenesis studies (step (a)) and computer simulations of the structure of the binding site provided that the precise three-dimensional structure of the antagonists or inhibitors is known (if not, it can be predicted by computational simulation). If said antagonist or inhibitor is itself a peptide, it can be also mutated to determine which residues interact with others in the above-mentioned polypeptide essential for bacterial growth and survival.

Finally, in step (c) the antagonist or inhibitor can be modified to improve its binding affinity or its potency and specificity. If, for instance, there are electrostatic interactions between a particular residue of an polypeptide as defined above and some region of an antagonist or inhibitor molecule, the overall charge in that region can be modified to increase that particular interaction. Furthermore, the three-dimensional and/or crystallographic structure of inhibitors or antagonists of the polypeptide of the invention can be used for the design of peptidomimetic inhibitors or antagonists, e.g. in combination with said polypeptide (Rose, Biochemistry 35 (1996), 12933-12944; Rutenber, Bioorg. Med. Chem. 4 (1996), 1545-1558).

Potential antagonists/inhibitors include antisense molecules. Antisense technology can be used to control gene expression through antisense DNA or through triple-helix formation. Antisense techniques are discussed, for example, in Okano, J. Neurochem. 56 (1991), 560; Oligodeoxynucleotides as Antisense Inhibitors of Gene Expression, CRC Press, Boca Raton, Fla. (1988). Triple helix formation is discussed in, for instance, Lee, Nucl. Acids Res. 6 (1979), 3073; Cooney, Science 241 (1988), 456; and Dervan, Science 251 (1991), 1360. The methods are based on binding of a polynucleotide to a complementary DNA or RNA.

For example, the 5′ coding portion of a polynucleotide that encodes the mature polypeptide as described above may be used to design an antisense RNA oligonucleotide of from about 10 to 40 base pairs in length. A DNA oligonucleotide is designed to be complementary to a region of the gene involved in transcription thereby preventing transcription and the production of the protein. The antisense RNA oligonucleotide hybridizes to the mRNA and blocks translation of the mRNA molecule into receptor polypeptide. As indicated, antagonist or inhibitor e.g. polyclonal and monoclonal antibody according to the teachings of the present invention can be raised according to the methods disclosed in Tartaglia, J. Biol. Chem. 267 (1992), 4304-4307; Tartaglia, Cell 73 (1993), 213-216, and PCT Application WO 94/09137.

Antibodies may be prepared by any of a variety of methods using immunogens of the polypeptide described above. As indicated, such immunogens include the full length polypeptide (which may or may not include the leader sequence) and fragments such as the ligand binding domain, the extracellular domain and the intracellular domain. These antibodies can be monoclonal antibodies, polyclonal antibodies or synthetic antibodies as well as fragments of antibodies, such as Fab+, Fv, F(ab′)₂, disulphide-bridged Fv or scFv fragments, etc. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988.

The antagonists or inhibitors isolated by the above methods also serve as lead compounds for the development of analog compounds. The analogs should have a stabilized electronic configuration and molecular conformation that allows key functional groups to be presented to the receptor in substantially the same way as the lead compound. In particular, the analog compounds have spatial electronic properties which are comparable to the binding region, but can be smaller molecules than the lead compound, frequently having a molecular weight below about 2 kD and preferably below about 1 kD. Identification of analog compounds can be performed through use of techniques such as self-consistent field (SCF) analysis, configuration interaction (CI) analysis, and normal mode dynamics analysis. Computer programs for implementing these techniques are available; e.g., Rein, Computer-Assisted Modeling of Receptor-Ligand Interactions (Alan Liss, New York, 1989). Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art. Furthermore, peptidomimetics and/or computer aided design of appropriate derivatives and analogues can be used, for example, according to the methods described above.

The inhibitor or antagonist identified by the above-described method may prove useful as a pesticide, and/or antibiotic. The inhibitors and antagonists of the present invention preferably have a specificity at least substantially identical to the binding specificity of the natural ligand or binding partner of the polypeptide described above. An antagonist or inhibitor can have a binding affinity to said polypeptide of at least 10⁵ M−1, preferably higher than 10⁷ M−1 and advantageously up to 10¹⁰ M⁻¹. In a preferred embodiment, an inhibitor, e.g. suppressive antibody, has an affinity of at least about 10⁻⁷ M, preferably at least about 10⁻⁹ M and most preferably at least about 10⁻¹¹ M; and the antagonist has an affinity of less than about 10⁻⁷ M, preferably less than about 10⁻⁹ M and most preferably in order of 10⁻¹¹ M.

In the case of nucleic acid molecules it is preferred that they have a binding affinity to those encoding the amino acid sequences encoded in any one of SEQ ID NOS: 16 to 39 of at most 2-, 5- or 10-fold less than an exact complement of 20 consecutive nucleotides of the above described nucleic acid molecules.

In another embodiment, the present invention relates to a method for producing a therapeutic agent comprising synthesizing the above-described antagonist or inhibitor.

Preferably, the compound identified according to the above described method or its analog or derivative is further formulated in a therapeutically active form or in a form suitable for the application against bacterial infections or diseases related to such an infection. For example, it can be combined with a pharmaceutically acceptable carrier known in the art. Thus, the present invention also relates to a method of producing a (therapeutically effective) composition comprising the steps of one of the above described methods of the invention and combining the compound obtained or identified in the method of the invention or an analog or derivative thereof with a pharmaceutically acceptable carrier.

Also, the present invention relates to a composition comprising the antagonist or inhibitor mentioned above. As is evident from the above, the present invention generally relates to compositions comprising at least one of the aforementioned antagonists or inhibitors, which may be nucleic acid molecules, proteins or antibodies. Advantageously, said composition is for use as a medicament, a diagnostic means, or a kit.

The term “composition”, as used in accordance with the present invention, comprises at least one small molecule or molecule as identified herein above, such as a protein, an antigenic fragment of said protein, a fusion protein, a nucleic acid molecule and/or an antibody as described above and, optionally, further molecules, either alone or in combination, like e.g. molecules which are capable of optimizing antigen processing, cytokines, immunoglobulins, lymphokines or CpG-containing DNA stretches or, optionally, adjuvants. The composition may be in solid, liquid or gaseous form and may be, inter alia, in form of (a) powder(s), (a) tablet(s), (a) solution(s) or (an) aerosol(s). In a preferred embodiment, said composition comprises at least two, preferably three, more preferably four, most preferably five differentially synthesized proteins.

The antagonists and inhibitors of the invention appear to function against gene products which are essential in several strains or genera of bacteria. Accordingly, the above-described antagonists and inhibitors may be used to inhibit the growth of a wide spectrum of bacteria. The above described antagonists or inhibitors may be used to slow, stop, or reverse bacterial growth. Thus, the present invention also relates to a method of producing a therapeutic agent comprising the steps of the methods described hereinbefore and synthesizing the antagonist or inhibitor obtained or identified as described above or an analog or derivative thereof, preferably in an amount sufficient to provide said agent in a therapeutically effective amount to a patient.

Compounds identified by the above methods or analogs are formulated for therapeutic use as pharmaceutical compositions. The compositions can also include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of protein or its antibodies, antagonists, or inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg per dose; however, doses below or above this exemplary range are envisioned, especially considering the aforementioned factors. Administration of the suitable compositions may be effected by different ways, e.g., by intravenous, intraperitoneal, subcutaneous, intramuscular, topical or intradermal administration. If the regimen is a continuous infusion, it should also be in the range of 1 μg to 10 mg units per kilogram of body weight per minute, respectively. Progress can be monitored by periodic assessment. The compositions of the invention may be administered locally or systemically. Administration will generally be parenterally, e.g., intravenously. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins, interferons and/or CpG-containing DNA stretches, depending on the intended use of the pharmaceutical composition.

In another embodiment, the present invention relates to a kit comprising at least one of the aforementioned antagonists or inhibitors of the invention. The kit of the invention as well as the composition may in a preferred embodiment contain further ingredients such as selection markers, antibiotics, cytokines and components for simplifying or supporting the treatment of bacterial infections or disorders or diseases related to bacterial infections. The kit of the invention may advantageously be used for carrying out the method of the invention and could be, inter alia, employed in a variety of applications referred to herein, e.g., in the diagnostic field or as research tool. The parts of the kit of the invention can be packaged individually in vials or in combination in containers or multicontainer units. Manufacture of the kit follows preferably standard procedures which are known to the person skilled in the art. The kit or its ingredients according to the invention can be used in antibacterial therapies, for example, for any of the above described methods for detecting further inhibitors and antagonists essential for bacterial growth and survival. The kit of the invention and its ingredients are expected to be very useful for the healing and protection of animals and humans suffering from a bacterial infection.

The present invention also relates to a method for treating or preventing bacterial infections or diseases or disorders related to bacterial infections comprising the step of administering to a subject in need thereof an antagonist or inhibitor identified herein above, optionally comprised in a pharmaceutical composition of the invention.

In another embodiment the present invention relates to the use of a polypeptide encoded by the gene as identified above or a fragment, derivative or ortholog thereof or of any of said genes for the identification of an antagonist or inhibitor of said polypeptide fragment, derivate or ortholog or said gene.

In a further embodiment the present invention relates to the use of said polypeptide, the therapeutic agent produced according to the invention, the antagonist or inhibitor obtained or identified by the method or use according to the invention for the preparation of a pharmaceutical composition for the treatment of (a) bacterial infection(s), disorder(s) and/or disease(s) related to bacterial infections.

In another embodiment the present invention relates to a method for treating or preventing bacterial infections or diseases or disorders related to bacterial infections comprising the step of administering to a subject in need thereof an antagonist or inhibitor identified herein above, optionally comprised in the pharmaceutical composition according to the present invention.

In a further embodiment the present invention relates to the use of the above-described polypeptide, a fragment, derivative or ortholog thereof or of any of said genes for screening for polypeptides interacting with said polypeptide using protein-protein interaction technologies, and/or for validating such interaction as being essential for bacterial survival and/or for screening for antagonists or inhibitors of such interaction.

In a further embodiment the present invention relates to the use of the above-described polypeptide, a fragment, derivative or ortholog thereof or of any of said genes for screening of polypeptide for polypeptide binding to said polypeptide, and/or for validating the peptides binding to said polypeptide as preventing growth of bacteria or being lethal to bacteria upon expression of said polypeptides in said bacteria, and/or for screening for small molecules competitively displacing said peptides.

In another embodiment the present invention relates to the use of a conditional mutant of a gene as described above or a fragment, derivative or ortholog thereof or of surrogate ligands against said gene expressed in bacteria to induce a lethal phenotype in bacteria and/or for the analysis of said bacteria for surrogate markers by comparison of RNA or protein profiles in said bacteria with RNA or protein profiles in wild type bacteria, and/or the use of said surrogate markers for the identification of antagonists of the essential function of said gene.

In another embodiment the present invention relates to a method for identifying or isolating a surrogate marker comprising the steps as described in the above-recited method of the present invention.

In a further embodiment the present invention relates to a method for identifying or isolating a surrogate marker comprising the steps of

(a) inducing a lethal phenotype in bacteria representing a conditional mutant of a gene selected from the group consisting of ygbB, yfhC, yacE, ychB, yejD, yrfl, yggJ, yjeE, yiaO, yrdC, yhbC, ygbP, ybeY, gcpE, kdtB, pfs, ycaj, b1808, yeaA, yagF, b1983, yidD, yceG and/or yjbC; and

(b) analysing said bacteria comparing the RNA or protein profile of said bacteria with wild type bacteria.

These and other embodiments are disclosed and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The present invention is further illustrated by reference to the following non-limiting examples.

Unless stated otherwise in the examples, all recombinant DNA techniques are performed according to protocols as described in Sambrook et al. (1989), Molecular Cloning: A Laboratory Manual. Cold Spring Harbor Laboratory Press, NY or in Volumes 1 and 2 of Ausubel et al. (1994), Current Protocols in Molecular Biology, Current Protocols. Standard materials and methods for plant molecular work are described in Plant Molecular Biology Labfase (1993) by R. D. D. Croy, jointly published by BIOS Scientific Publications Ltd. (UK) and Blackwell Scientific Publications (UK).

### BRIEF DESCRIPTION OF THE FIGURES

FIG. 1: Sequences of the essential bacterial genes identified according to the method described in the examples.

FIG. 2: PCR strategy and the position of primers used.

FIG. 3: Sequence comparison table of essential E. coli genes with proposed orthologs from various bacteria. Unfinished genomes are indicated by asterisk. Complete genomes were analysed using BlastP2. Unfinished genomes were analysed with TBlastN. Orthologous sequences can be accessed at the respective WWW links as indicated in the footnotes.

FIG. 4: Multiple Sequence Alignment (MSA) of E. coli gene ygbB with orthologs in 5 different bacterial organisms including homology score. Similar MSA with similar results have been created for all 22 essential bacterial genes.

### EXAMPLE 1

An automated BLASTP-based genome comparisons to identify E. coli FUN genes resulted in the following list of 65 candidate genes which are conserved between E. coli, B. subtilis, H. influenzae, H. pylori, M. tuberculosis, Ch. trachomatis, B. burgdorferi, T. pallidum, S. pneumoniae, S. aureus, E. faecalis, P. aeruginosa, B. pertussis and which were further analysed:

| | Gene | | Gene | | |
|---|---|---|---|---|---|
| | Bank | | Bank | | Gene Bank |
| FUN | Accession | FUN | Accession | FUN | Accession |
| Genes | Number | Genes | Number | Genes | Number |
| ygbB | g1789103 | yggS | g1789321 | yaeE | g1786397 |
| yhaD | g1789512 | yggV | g1789324 | yicC | g1790075 |
| yhbU | g1789548 | yggW | g1789325 | yebK | g1788159 |
| yhiN | g2367234 | yjhG | g2367371 | yhbC | g1789561 |
| yieG | g1790150 | yjiR | g1790797 | ygbP | g1789104 |
| yihZ | g1790320 | yohI | g1788462 | ybaX | g1786648 |
| yjgF | g1790691 | yqhThom | g1788728 | yqcD | g1789158 |
| yacE | g1786292 | yfiH | g1788945 | ybeY | g1786880 |
| yaeC | g1786396 | yhaR | g1789501 | gcpE | g1788863 |
| yagF | g1786464 | yhdG | g1789660 | kdtB | g1790065 |
| ybeB | g1786856 | yccG | g1787197 | pfs | g1786354 |
| ycfH | g147382 | ychB | g1787459 | sms | g1790850 |
| ydcP | g1787705 | yejD | g1788510 | ycaJ | g1787119 |
| ydiB | g1787983 | yidD | g140861 | yhhF | g1789875 |
| yebI | g1788166 | yrfI | g1789804 | yleA | g1786882 |
| yeeC | g1788320 | yggJ | g1789315 | b1808 | g1788110 |
| yegQ | g1788397 | yjeE | g1790610 | yeaA | g1788077 |
| yfcB | g1788670 | yiaO | g1790004 | b1675 | g1787964 |
| yfgB | g1788865 | yrdC | g2367210 | yhbU/yegQa | g1789548/ |
| | | | | | g1788397 |
| yfhC | g1788911 | b1983 | g1788294 | yjgF/yhaRa | g1790691/ |
| | | | | | g1789501 |
| ydiD | g1787993 | yeeS | g1736671 | b2385 | g1788728 |
| nlpA | g72589 | yaaJ | g1786188 | yicO | g1790097 |
| yfjY | g1788997 | ydhE | g1742737 | yebC | g140614 |
| ykfG | g2367100 | yjcD | g396399 | yohI/yhdGa | g1788462/ |
| | | | | | g1789660 |
| ygcA | g1789148 | yceG | g1787339 | smpB | g1788973 |
| ygfA | g1789278 | yjbC | g396357 | | |

a: double mutants were created when the respective genes were paralogues in E. coli

Creating In-Frame Deletions of E. coli Genes

The subsequent description of the construction of deletion mutants was carried out essentially equal for these 77 candidate genes. Particular details will exemplarily be described for one gene which gave rise to be essential (yfhC) and one which was non-essential (yggV).

1) Principle of the PCR-Procedure and Primer-Design for in Frame Deletions:

Unless an overlapping ORF exists, primers dgenX2 and dgenX3 are designed to delete the entire ORF from ATG to STOP, e.g.: ATGgttataaatttggagtgtgaaggttattgcgtgTAA (SEQ ID NO: 1) (see figure). The 5′-ends of primers dgenX1 and dgenX4 contain random nucleotides followed preferably by a BamHI site (dgenx1) or a SalI site (dgenX4) for cloning into plasmid pKO3 (Link et al (1997), J Bac 179: 6228-6237). In most mutants, primers dgenX2 and dgenX3 contain a 33 bp tag sequence called “Church-tag”.

Church-tag forward direction: 5′-gttataaatttggagtgtgaaggttattgcgtg-3′ (SEQ ID NO: 2)

Church-tag reverse direction: 5′-cacgcaataaccttcacactccaaatttataac-3′ (SEQ ID NO: 3)

This tag is used for a subsequent PCR in which the 5′- and 3′-flanking DNA-fragments of the deletion construct are assembled.

In the few constructs lacking the “Church-tag”, the primers dgenX2 and dgenX3 carry at their 5′-ends 5 random nucleotides followed by a restriction site (preferably EcoRI) which by its positioning creates the in frame deletion.

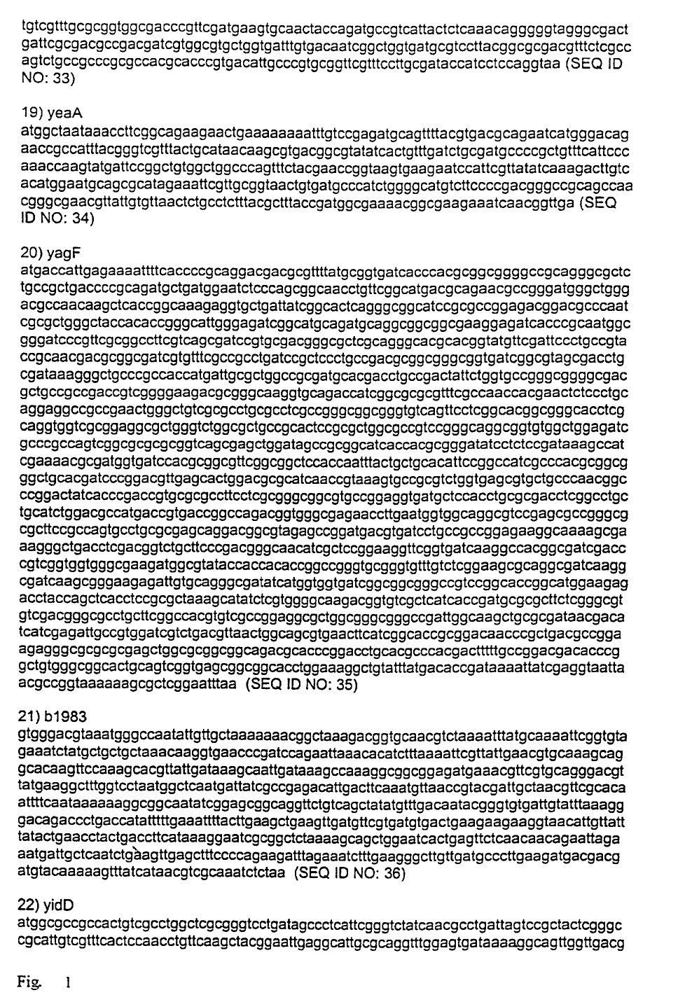

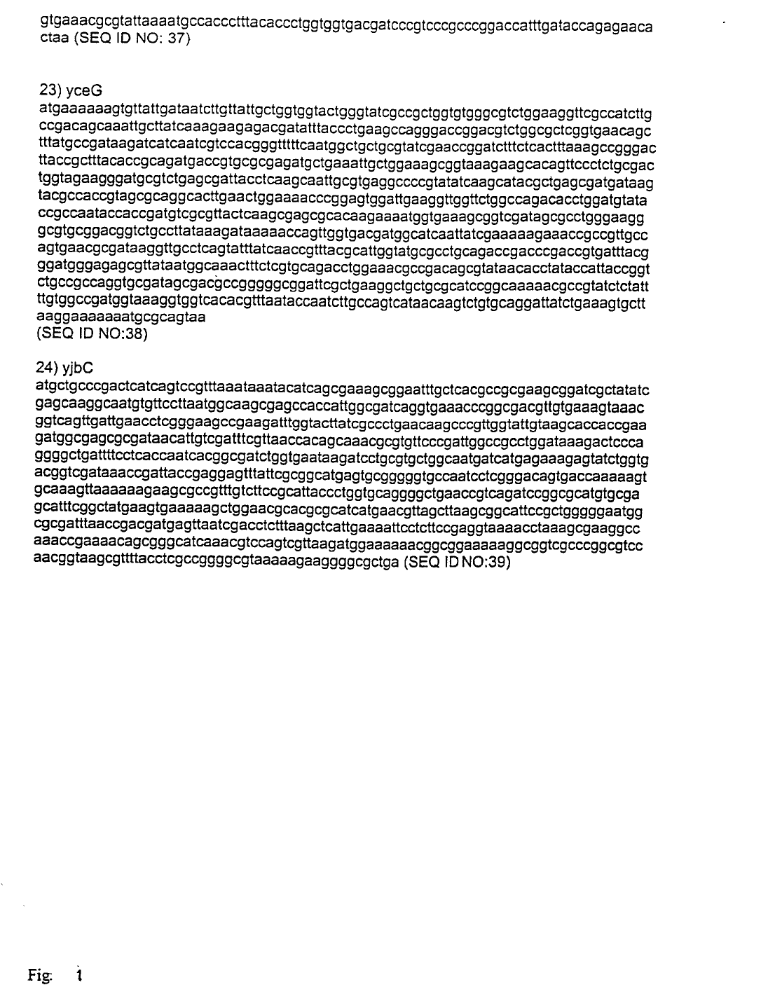

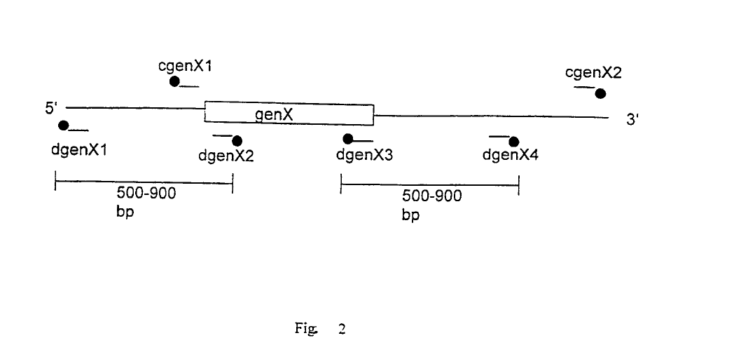

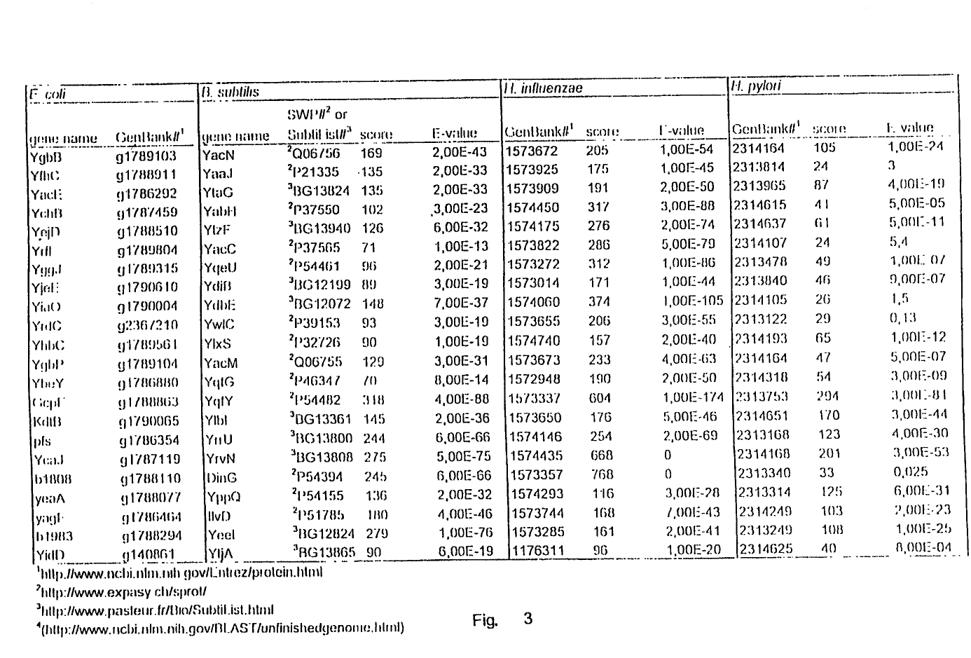

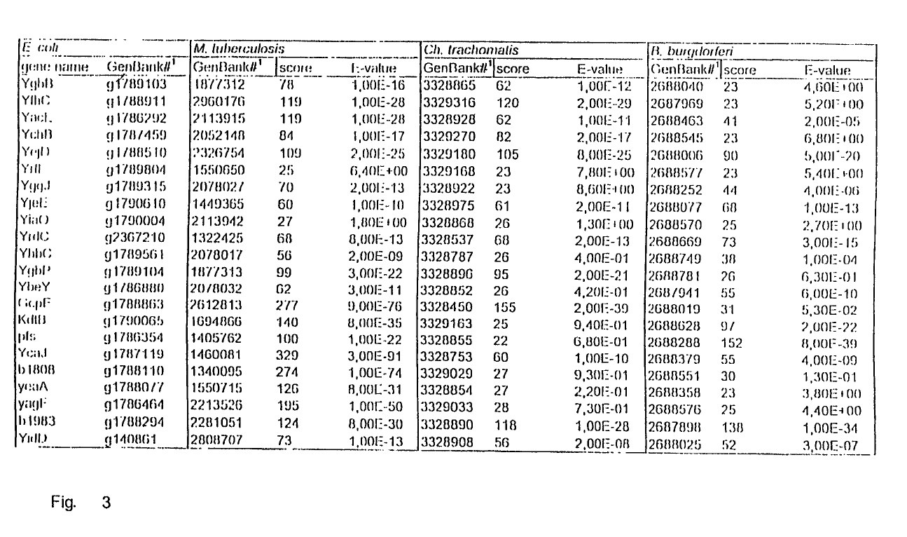

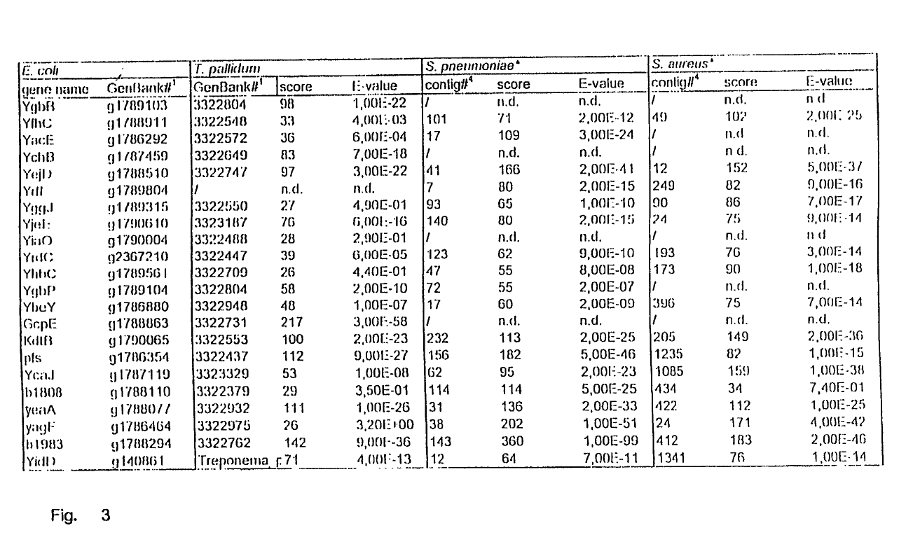

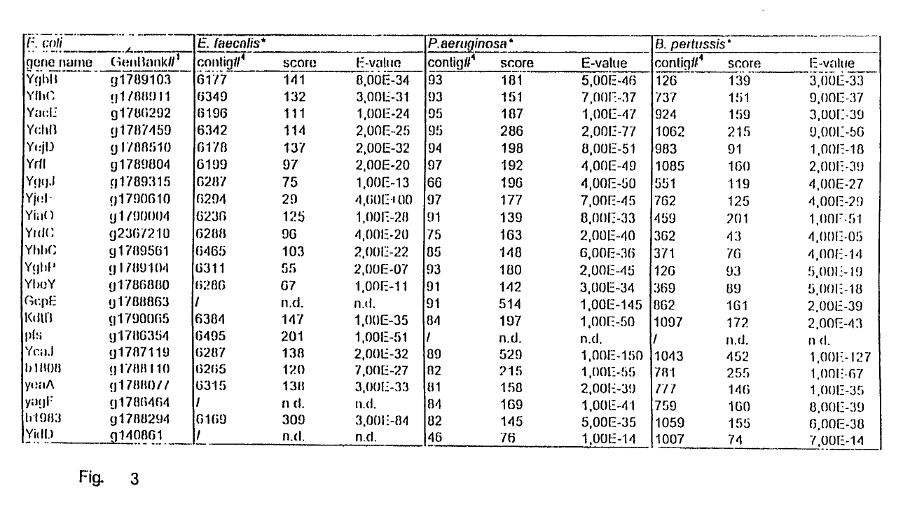

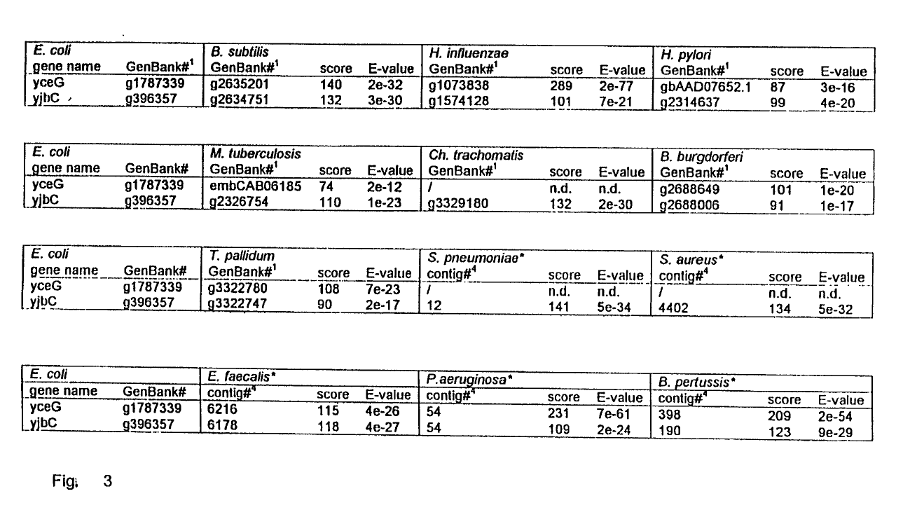

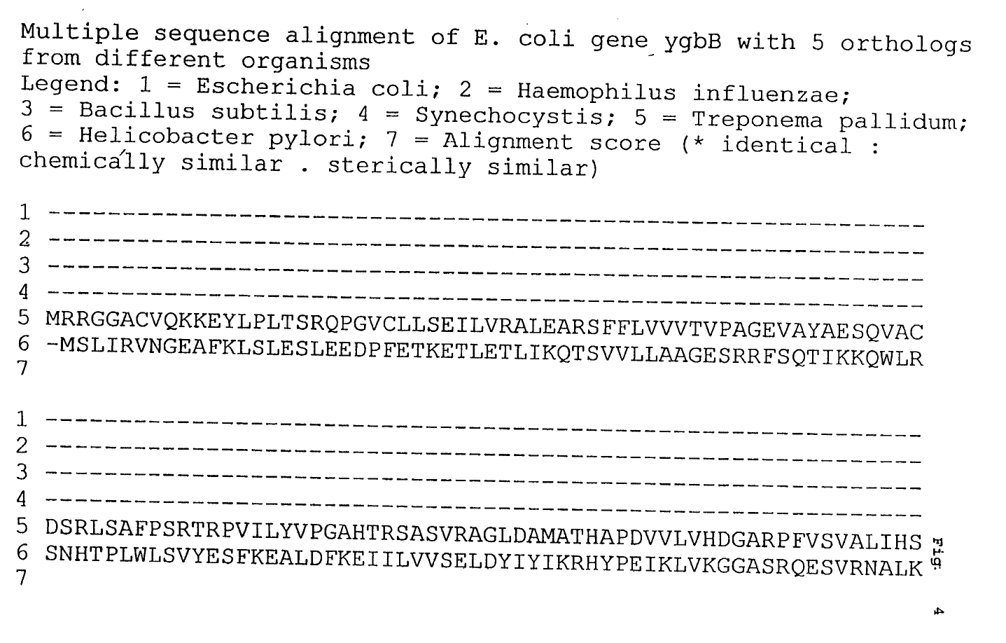

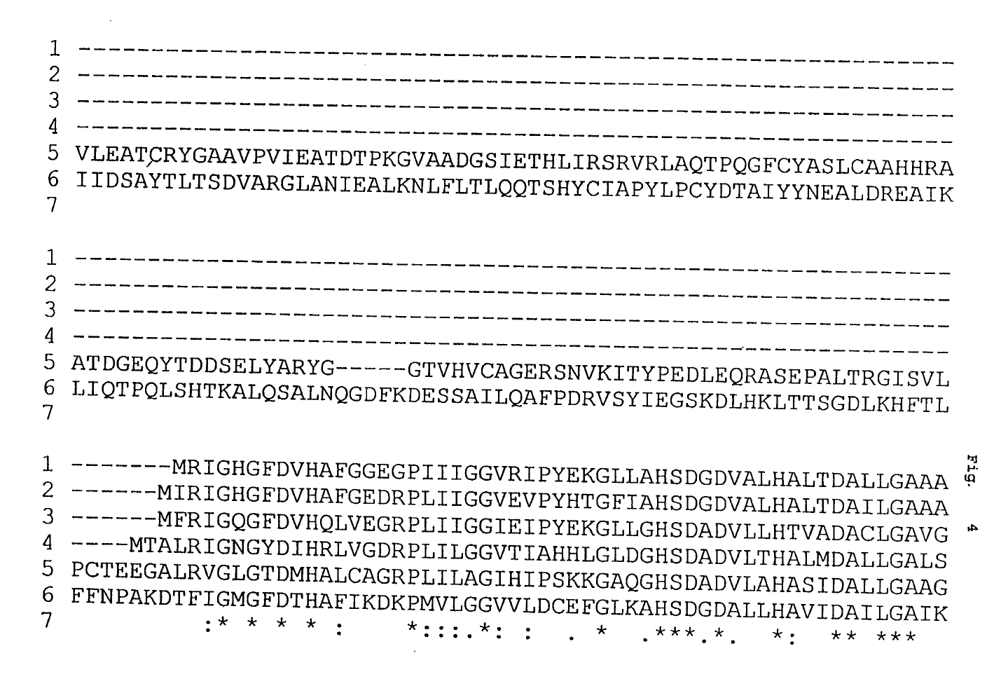

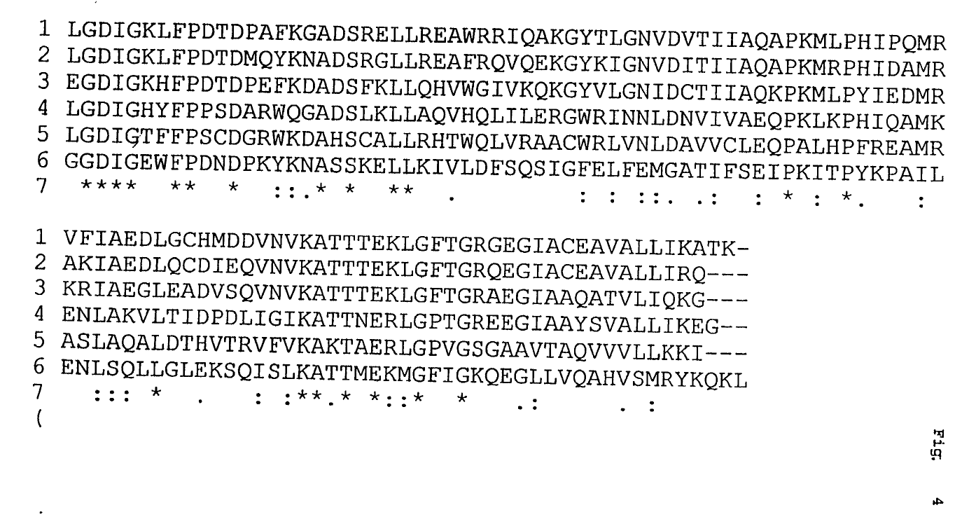

Oligos cgenX1 and cgenX2 are used for the verification of the chromosomal situation (wild type or deletion) after the replacement procedure (FIG. 2).

Primers for the respective candidate genes were designed as follows:

dyfhC1: 5′-GATCGGATCCAAATTCCAGTTAGCCATGATGCGGTC-3′ (SEQ ID NO: 4)

dyfhC2: 5′-CACGCAATAACCTTCACACTCCAAATTTATAACCATTATACACGGACG CTATGC-3′ (SEQ ID NO: 5)

dyfhC3: 5′-GTTATAAATTTGGAGTGTGAAGGTTATTGCGTGACGGATTAATTTTGTT TCTCTT-3′ (SEQ ID NO: 6)

dyfhC4: 5′-GATCGTCGACGCGCTCGATATCACCGATGAACAACCG-3′ (SEQ ID NO: 7)

cyfhC1: 5′-CAATCCGCTGCTTTATTTCTGTCAG-3′ (SEQ ID NO: 8)

cyfhC2: 5′-TTATAACGAAATCAACGGGAAACCT-3′ (SEQ ID NO: 9)

dyggV1: 5′-GATCGGATCCCTCTAAAAAATAAGGAATTAAAGG-3′ (SEQ ID NO: 10)

dyggV2: 5′-CACGCAATAACCTTCACACTCCAAATTTATAACCATAGGATACCTAAT TAATTAAC-3′ (SEQ ID NO: 11)

dyggV3: 5′-GTTATAAATTTGGAGTGTGAAGGTTATTGCGTGAAGAGCGCCATTTCC CACCGT-3′ (SEQ ID NO: 12)

dyggV4: 5′-GATCGTCGACTCATATTGCTGATAACCCGCTGCGGT-3′ (SEQ ID NO: 13)

cyggV1: 5′-GTTGACGGCCAGGCCAACAGTCAT-3′ (SEQ ID NO: 14)

cyggV2: 5′-ATAACCCTGGGCAATCGCCTCG-3′ (SEQ ID NO: 15)

### EXAMPLE 2

### Construction of the DNA-Fragments Comprising the Deletion

The 5′- and the 3′-Flanking DNA Fragments are PCR Amplified in a Total Volume of 50 μl as Follows:

| | |
|---|---|
| Chromosomal DNA from E. coli strain | final conc.: 1 ng/μl |
| MG1655 (100 ng/μl): | |
| 10*Pwo-buffer | final conc.: 1x |
| dgenX1/3 (10 μM) | final conc.: 500 nM |
| dgenX2 (4) (10 μM) | final conc.: 500 nM |
| Pwo-Polymerase | final conc.: 5 U/100 μl |
| dNTPs (25 mM) | final conc.: 250 μM |
| H₂O | to adjust volume to 50 μl |

PCR Conditions:

4′ 94° C.

30 cycles: 30″ 94° C., 30″ 44° C., 1′ 72° C.

5′ 72° C.

The PCR products are then purified with the High Pure PCR Purification Kit (Boehringer) to remove salts and enzyme (elute in 50 μl H₂O). Alternatively, if PCR products contain prominent impurities, the respective fragment must be purified by agarose gel extraction (Gene Clean, Dianova) before the fragment assembly.

Assembly PCR

Equal amounts of 5′- and 3′-fragment are applied as template DNA. In general a volume applied for gel electrophoresis giving an intense band is o.k. The total reaction volume is 100 μl. For the assembly the “outer” primers dgenX1 and dgenX4 were used.

| | |
|---|---|
| 5′-Fragment | approx. 10 ng |
| 3′-Fragment | approx. 10 ng |
| 10*Pwo-buffer | final conc.: 1x |
| dgenX1 (10 μM) | final conc.: 500 nM (50 pmol/100 μl) |
| dgenX4 (10 μM) | final conc.: 500 nM (50 pmol/100 μl) |
| Pwo-Pol (Boehringer) | final conc.: 5 U/100 μl |
| dNTPs (25 mM) | final conc.: 250 μM |
| H₂O | add to 100 μl |

PCR Conditions:

4′ 94° C.

10 cycles: 30″ 94° C., 30″ 44° C., 1′72° C.

25 cycles: 30″ 94° C., 30″ 44° C., 3′ 72° C.

5′ 72° C.

The success of the PCR is checked by agarose gel electrophoresis. The assembled PCR product is purified with the High Pure PCR Purification Kit and the complete eluate of 50 μl is overnight digested with BamHI and SalI in a volume of 60 μl. After gel electrophoresis the digested product is purified with Gene Clean (Dianova) to remove small oligonucleotides quantitatively (elution volume: 25 μl).

Cloning into Vector pKO3:

Next, the fragment is ligated into the vector pKO3 (cut with BamHI and SalI) in a 10-20 ml reaction (T4-DNA ligase) for 2 hours at room temperature.

Transformation into DH5:

One half of the ligation mix is transformed into chemically competent E. coli DH5a and clones are purified once (usually 8 clones are sufficient).

Verification of Deletion Constructs:

1) 8 clones are characterized by colony-PCR with vector pKO3-specific primers (pKO3-B1 and pKO3-S1).

2) Clones with the correct size of insert are double-checked by colony-PCR with gene specific primers (dgenX1 and dgenX4).

Reaction Mixture for 25 μl Reaction Volume:

| | |
|---|---|
| template (colony) | 1 μl of 1 colony resuspended in 20 μl H₂O |
| 10*Taq-buffer | final conc.: 1x |
| 5*Q-solution | final conc.: 1x |
| pKO3-B1/dgenX1 (100 μM) | final conc.: 1 μM (50 pmol/100 μl) |
| pKO3-S1/dgenX4 (100 μM) | final conc.: 1 μM (50 pmol/100 μl) |
| Taq-Pol (QIAgen) | final conc.: 2 U/25 μl |
| dNTPs (25 mM) | final conc.: 250 μM |
| H₂O | 15.35 μl |

PCR Conditions:

4′ 94° C.

25 cycles: 30″ 94° C., 30″ 50° C., 2′ 65° C.

5′ 65° C.

3) Plasmid-DNA from 4 ml over-night culture is prepared using a QIAgen Miniprep Kit and a double restriction analysis with BamHI/SalI and EcoRI/HindIII is performed to verify the clones.

Protocol Referring to the Construction of Assembly Products by a Restriction Site:

The 5′- and the 3′-fragments are PCR amplified as described above. The PCR products are purified with the High Pure PCR Purification Kit (Boehringer) to remove salts and enzyme and 5 to 10 μl are digested over night using the restriction site creating the deletion (primers 2 and 3; mostly EcoRI) in a total volume of 30 μl. The restriction products are again purified with the High Pure PCR Purification Kit to remove nucleotides, salts and enzyme. (Alternatively: Following preparative agarose gel electrophoresis the cut fragments are isolated using Gene Clean (Dianova) and eluted in a volume of 25 μl. The cut fragments (3-6 μl each) are ligated in a volume of 10-15 μl using T4-DNA ligase for 2 hours at room temperature. 5 μl of this ligation mix is directly used as a template for a second PCR. In this PCR, the assembled fragments are amplified using primers dgenX1 and dgenX4. The reaction is set up as described above with two exceptions: 1) The total reaction volume is 100 μl and 2) the extension step at 72° C. lasts 3′.

### EXAMPLE 3

### The Chromosomal Exchange Strategy

(Link et al (1997), J Bac 179: 6228-6237)

Cointegration:

Cointegration=integration of a plasmid into the chromosome by a recombination event

The pKO3 derivative is transformed into MG1655 or any recA⁺ strain

Day 1

The strain is grown at 30° C. in LB containing 20 μg/ml chloramphenicol (LB-Cam2O) to an OD₆₀₀ of ˜1.0. Afterwards, perform 10-fold serial dilutions in the same medium (down to 10⁻⁷). For the following plating use prewarmed LB-Cam20 agar plates. Plate 100 μl of dilutions 10⁻⁴ and 10⁻⁵ for incubation at 44° C. and 100 μl of dilutions 10⁻⁶ and 10⁻⁷ for incubation at 30° C.

Day 2

Following incubation at the respective temperature, determine the factor c.f.u.44° C./c.f.u.30° C. (c.f.u.=colony forming units). This factor for pKO3 without insert is in the range 1×10⁻⁴ to 5×10⁻⁴ and should be significantly larger in the case of successful cointegration. Purify 8 randomly chosen clones from the 44° C. plate twice on LB-Cam20 agar plates at 44° C. (during Day 2 and over night to Day 3). Optionally, confirm the clones for their identity as cointegrates by colony-PCR.

Resolution and Counter-Selection:

Resolution=resolution of the cointegrate resulting in a self replicative plasmid by a second recombination event

Counter-selection=selection against the presence of plasmid in the cell

Day3

Pool single colonies from each of the 8 cointegrates in 100 μl LB and use this suspension as an inoculum for 10 ml LB+5% sucrose. After growth at 30° C. (8 to 10 hours during a day is sufficient) 10-fold serial dilutions are performed and 100 μl of dilutions 10⁻⁴, 10⁻⁵, and 10⁻⁶ are plated onto LB agar+5% sucrose and grown over night at 30° C.

Day 4

50 single colonies are replica streaked on LB+Cam20 and LB+5% sucrose to test for the loss of plasmid.

### EXAMPLE 4

### Testing for Essentiality of FUN Genes of E. coli and Interpretation of the Results

Day 5

The clones sensitive to chloramphenicol are then tested for their genotype (wild type versus in-frame deletion) by colony-PCR using primers cgenX1 and cgenX2 (10-48 clones).

In the case of the gene yfhC out of 48 clones tested only wild type situation on the chromosome could be detected.

In the case of the gene yggV out of 48 clones 16 (=33%) revealed a PCR product with a size indicative for the deletion situation on the chromosome.

Are 48 clones revealing no mutant enough to claim a gene as essential? This question can be answered by asking for the number of clones that have to be tested to get a confidence of e.g. 99% that really no mutants are present in an infinite number of clones. Provided a hypothesis H₀ means that only the wild type genotype is viable and hypothesis H₁ means that a fraction (1−x) of mutants is allowed to occur together with the wild type (x) among a population of clones (x+(1×)), then the probability to make the wrong decision (decision for H₀ whereas H₁ is true) can be calculated as

xⁿ/(1+xⁿ) (1)

where x is the fraction of wild type clones and n is the number of clones tested. The confidence niveau a to make the wrong decision (error probability) is given by

a>xⁿ/(1+xⁿ) (2)

thereby resulting in

n>ln(α/(1−α)/ln(x) (3)

for the number of clones that have to be tested to prove or disprove hypothesis H₀.

If the average probability for obtaining wild type clones (x) in a replacement experiment is 70% (experimentally determined for 43 non-essential genes out of 65 candidate genes), then, after testing of 26 clones which reveal a wild type genotype an uncertainty of 0.01% error probability (α) remains that the claiming of a gene as essential could be wrong. Even if the rate of obtaining wild types (x) is set to 85% (a value which occurs with a frequency of 10% for replacement experiments with non-essential genes), then, by testing 32 clones (which was performed in every experiment giving rise to an essential gene) an error probability of only 0.6% remains to chose the wrong hypothesis.

### EXAMPLE 5

### List of Essential FUN Genes Obtained

By the described method the following 24 genes were obtained which gave no deletion genotype and are therefore claimed to be essential:

E. coli

| gene name | GenBank# |
|---|---|
| ygbB | g1789103 |
| yfhC | g1788911 |
| yacE | g1786292 |
| ychB | g1787459 |
| yejD | g1788510 |
| yrfI | g1789804 |
| yggJ | g1789315 |
| yjeE | g1790610 |
| yiaO | g1790004 |
| yrdC | g2367210 |
| yhbC | g1789561 |
| ygbP | g1789104 |
| ybeY | g1786880 |
| gcpE | g1788863 |
| kdtB | g1790065 |
| pfs | g1786354 |
| ycaJ | g1787119 |
| b1808 | g1788110 |
| yeaA | g1788077 |
| yagF | g1786464 |
| b1983 | g1788294 |
| yidD | g140861 |
| yceG | g1787339 |
| yjbC | g396357 |

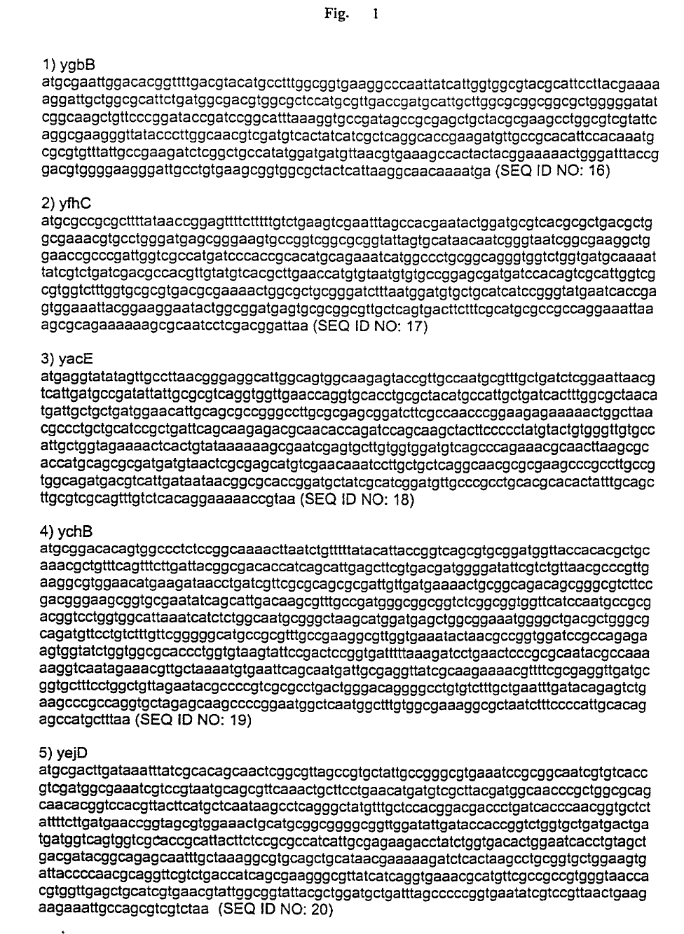

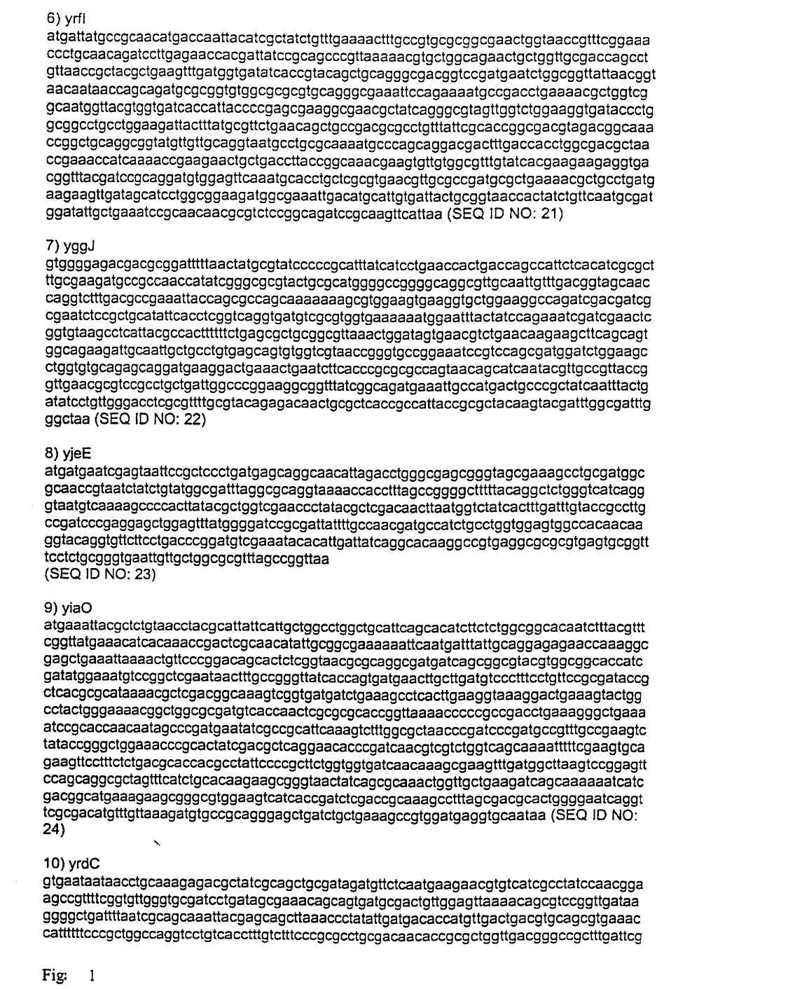

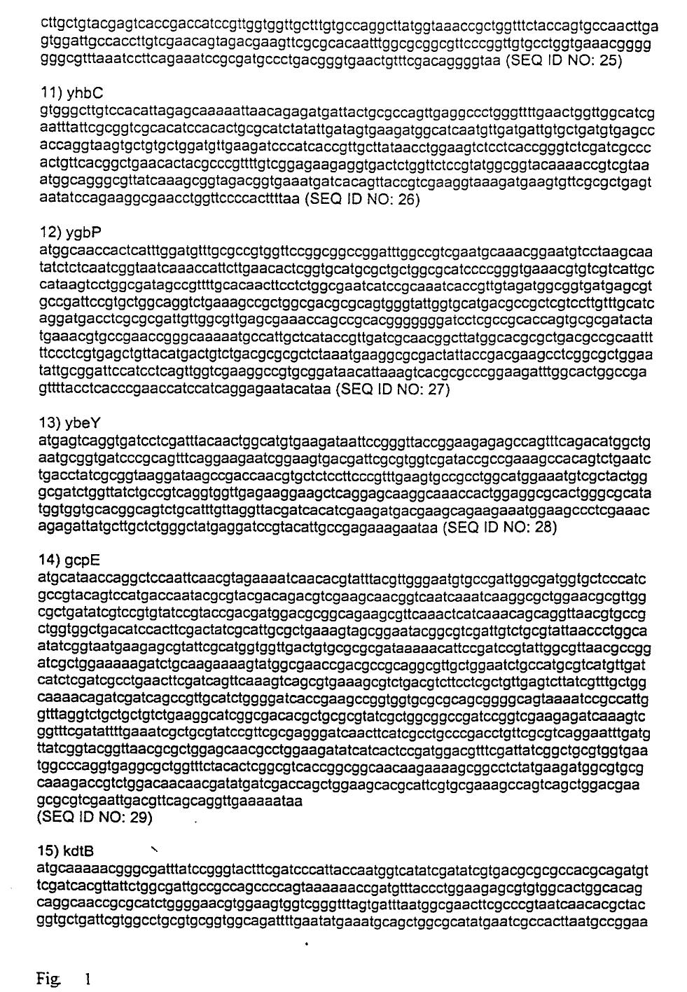

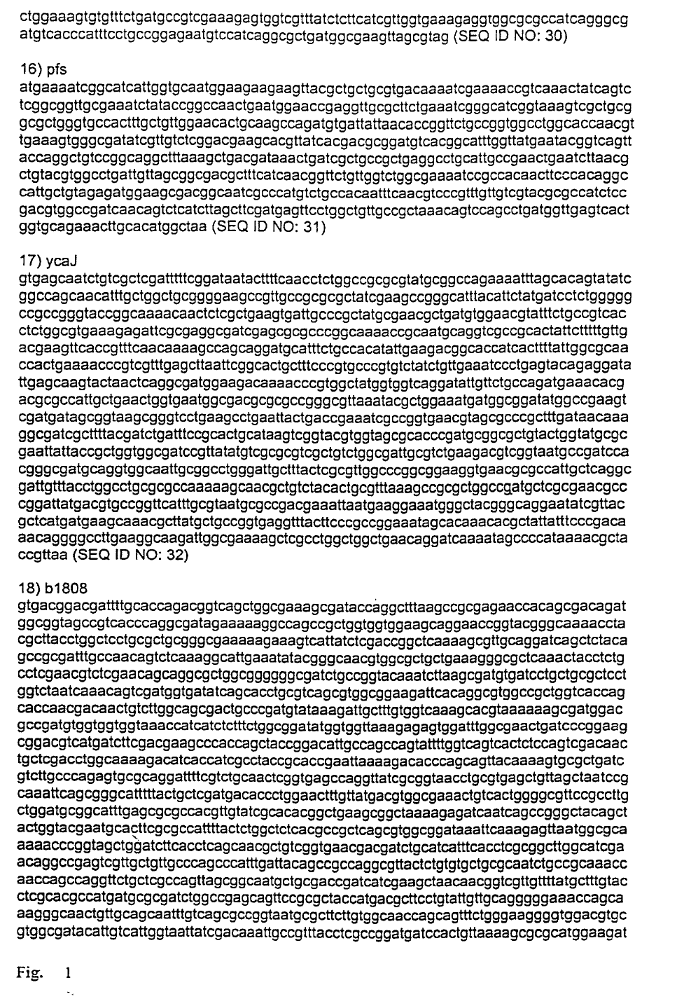

1. A method for identifying an antagonist or inhibitor of the expression of a gene encoding a polypeptide essential for bacterial growth or survival wherein said gene is selected from the group consisting of ygbB, yfhC, yacE, ychB, yejD, yrfl, yggJ, yjeE, yiaO, yrdc, yhbC, ygbP, ybeY, gcpE, kdtB, pfs, ycaj, b1808, yeaA, yagF, b1983, yidD, yceG and/or yjbC, the sequence of said genes being shown in FIG. 1, or a fragment, derivative or ortholog thereof, said method comprising the steps of
(a) testing a candidate antagonist or inhibitor or a sample comprising a plurality of said candidate antagonists or inhibitors for the inhibition or reduction of transcription of said gene or a fragment or derivative thereof, or
(b) testing a candidate antagonist or inhibitor or a sample comprising a plurality of said candidate antagonists or inhibitors for the inhibition or reduction of translation of mRNA transcribed from said gene or a fragment or derivative thereof; and
(c) identifying an antagonist or inhibitor or a sample comprising a plurality of said candidate antagonists or inhibitors that tests positive in step (a) and/or (b).

2. A method for testing a candidate antagonist or inhibitor or sample of a polypeptide or a mRNA essential for bacterial growth or survival encoded by a gene selected from the group consisting of ygbB, yfhC, yacE, ychB, yejD, yrfl, yggJ, yjeE, yiao, yrdc, yhbC, ygbP, ybeY, gcpE, kdtB, pfs, ycaJ, b1808, yeaA, yagF, b1983, yidD, yceG and/or yjbC, the sequence of said genes being shown in FIG. 1, or a fragment, derivative or ortholog thereof, comprising the steps of:
(a) contacting a bacterial cell with said candidate antagonist or inhibitor or sample; and
(b) determining whether said contacting leads to cell growth inhibition and/or cell death resulting from eliminating the function of at least one of said genes.

3. A method for testing a candidate antagonist or inhibitor of the function of a gene essential for bacterial growth or survival wherein said gene is selected from the group consisting of ygbB, yfhC, yacE, ychB, yejD, yrfl, yggJ, yjee, yiaO, yrdC, yhbC, ygbP, ybeY, gcpE, kdtB, pfs, ycaj, b1808, yeaA, yagF, b1983, yidD, yceG and/or yjbC, the sequence of said genes being shown in FIG. 1, or a fragment, derivative or ortholog thereof, comprising the steps of
(a) contacting a bacterial cell comprising said gene with a candidate antagonist or inhibitor or a sample comprising a plurality of said candidate antagonists or inhibitors; and
(b) determining whether said contacting leads to cell growth inhibition and/or cell death resulting from eliminating the function of at least one of said genes.

4. The method of any one of claims 1 to 3 further comprising identifying an antagonist or inhibitor, optionally from said sample of candidate antagonists or inhibitors.

5. The method of any one of claims 1 to 4 wherein said inhibitor or antagonist is further improved by peptidomimetics or by applying phage display or combinatorial library technique step(s).

6. A method for designing an improved antagonist or inhibitor for the treatment of a bacterial infection or disorder or disease related to a bacterial infection comprising the steps
(a) identification of the binding site of an antagonist or inhibitor to the polypeptide ygbB, yfhC, yacE, ychB, yejD, yrfl, yggJ, yjeE, yiaO, yrdC, yhbC, ygbP, ybeY, gcpE, kdtB, pfs, ycaJ, b1808, yeaA, yagF, b1983, yidD, yceG and/or yjbC, the sequence of said genes being shown in FIG. 1, or obtained by or identified by the method of any one of claims 1 to 5 by site-directed mutagenesis and chimeric polypeptide studies;
(b) molecular modeling of both the binding site of said antagonist or inhibitor and the structure of said polypeptide; and
(c) modification of said antagonist or inhibitor to improve its binding specificity or affinity for the polypeptide.

7. An antagonist or inhibitor of the activity of a polypeptide encoded by a gene selected from the group consisting of ygbB, yfhC, yacE, ychB, yejD, yrfl, yggj, yjeE, yiaO, yrdC, yhbC, ygbP, ybeY, gcpE, kdtB, pfs, ycaj, b1808, yeaA, yagF, b1983, yidD, yceG and/or yjbC, the sequence of said genes being shown in FIG. 1, or fragment, derivative or ortholog thereof or of the expression of a gene encoding said polypeptide or said fragment, derivative or ortholog or obtained by or identified by the method of any one of claims 1 to 6.

8. A method for producing a therapeutic agent comprising synthesizing the antagonist or inhibitor identified, tested or designed according to the method of any one of claims 1 to 6 or the antagonist or inhibitor of claim 7 or an analog or derivative thereof.

9. A method for producing a composition comprising the steps of the method of any one of claims 1 to 6 or synthesizing the antagonist or inhibitor of claim 7 and formulating said inhibitor or antagonist in a pharmaceutically acceptable form.

10. A composition comprising an antagonist or inhibitor of claim 7, the therapeutic agent produced by the method of claim 8 or the antagonist or inhibitor obtained by or identified in the method of any one of claims 1 to 6 or produced according to claim 9 and optionally a pharmaceutically acceptable carrier.

11. The composition of claim 10 which is a pharmaceutical composition.

12. The composition of claim 10 which is a kit.

13. The composition of any one of claims 10 to 12 further comprising an antibiotic and/or cytokine.

14. Use of a polypeptide encoded by a gene selected from the group consisting of ygbB, yfhC, yacE, ychB, yejD, yrfl, yggJ, yjeE, yiaO, yrdC, yhbC, ygbP, ybeY, gcpE, kdtB, pfs, ycaj, b1808, yeaA, yagF, b1983, yidD, yceG and/or yjbC, the sequence of said genes being shown in FIG. 1, or a fragment, derivative or ortholog thereof or of any of said genes for the identification of an antagonist or inhibitor of the activity of said polypeptide or said fragment, derivative or ortholog or of the expression of a gene encoding said polypeptide or said fragment, derivative or ortholog.

15. Use of an antagonist or inhibitor of claim 7, the therapeutic agent produced by the method of claim 8 or the antagonist or inhibitor obtained by or identified in the method of any one of claims 1 to 6 or produced according to claim 9 or identified by the use of any of the claims for the preparation of a pharmaceutical composition for the treatment of (a) bacterial infection(s), disorder(s) and/or disease(s) related to bacterial infections.

16. A method for treating or preventing bacterial infections or diseases or disorders related to bacterial infections comprising the step of administering to a subject in need thereof the antagonist or inhibitor obtained by or identified in the method of any one of claims 1 to 6 or produced according to claim 9 optionally comprised in the pharmaceutical composition according to claim 11.

17. Use of a polypeptide encoded by a gene selected from the group consisting of ygbB, yfhC, yacE, ychB, yejD, yrfl, yggJ, yjeE, yiaO, yrdC, yhbC, ygbP, ybeY, gcpE, kdtB, pfs, ycaJ, b1808, yeaA, yagF, b1983, yidD, yceG and/or yjbC, the sequence of said genes being shown in FIG. 1, or a fragment, derivative or ortholog thereof or any of said genes for screening for polypeptides interacting with said polypeptide using protein-protein interaction technologies, and/or for validating such interaction as being essential for bacterial survival and/or for screening for antagonists or inhibitors of such interaction.

18. Use of a polypeptide encoded by a gene selected from the group consisting of ygbB, yfhC, yacE, ychB, yejD, yrfl, yggJ, yjeE, yiaO, yrdc, yhbC, ygbP, ybeY, gcpE, kdtB, pfs, ycaj, b1808, yeaA, yagF, b1983, yidD, yceG and/or yjbC, the sequence of said genes being shown in FIG. 1, or a fragment, derivative or ortholog thereof or any of said genes for screening of polypeptide for polypeptide binding to said polypeptide, and/or for validating the peptides binding to said polypeptide as preventing growth of bacteria or being lethal to bacteria upon expression of said polypeptides in said bacteria, and/or for screening for small molecules competitively displacing said peptides.

19. Use of conditional mutants in a gene selected from the group consisting of ygbB, yfhC, yacE, ychB, yejD, yrfl, yggJ, yjeE, yiaO, yrdC, yhbC, ygbP, ybeY, gcpE, kdtB, pfs, ycaj, b1808, yeaa, yagF, b1983, yidD, yceG and/or yjbC, the sequence of said genes being shown in FIG. 1, or a fragment, derivative or ortholog thereof or of surrogate ligands against said gene expressed in bacteria to induce a lethal phenotype in bacteria and/or for the analysis of said bacteria for surrogate markers by comparison of RNA or protein profiles in said bacteria with RNA or protein profiles in wild type bacteria, and/or the use of said surrogate markers for the identification of antagonists of the essential function of said gene.

20. A method for identifying or isolating a surrogate marker comprising the steps as described in claim 19.

21. A method for identifying or isolating a surrogate marker comprising the steps of
(a) inducing a lethal phenotype in bacteria containing a conditional mutant of a gene selected from the group consisting of ygbB, yfhC, yacE, ychB, yejD, yrfl, yggJ, yjeE, yiaO, yrdc, yhbC, ygbP, ybeY, gcpE, kdtB, pfs, ycaj, b1808, yeaA, yagF, b 1983, yidD, yceG and/or yjbC, the sequence of said genes being shown in FIG. 1; and
(b) analysing said bacteria comparing the RNA or protein profile of said bacteria with wild type bacteria.
